(19) United States
(12) Patent Application Publication
(10) Pub. No.: US 2015/0064115 A1
(43) Pub. Date: Mar. 5, 2015

(54) NEUROPHILIC NANOPARTICLES

(71) Applicant: University of Florida Research Foundation, Inc., Gainesville, FL (US)

(72) Inventors: Ana M. Ashizawa, Gainesville, FL (US); Lucia Notterpek, Gainesville, FL (US); Tetsuo Ashizawa, Gainesville, FL (US)

(21) Appl. No.: 14/390,584

(22) PCT Filed: Mar. 6, 2013
(86) PCT No.: PCT/US2013/029296

§ 371 Date: Oct. 3, 2014

Related U.S. Application Data
(60) Provisional application No. 61/620,600, filed on Apr. 5, 2012.
(60) Provisional application No. 61/697,492, filed on Sep. 6, 2012.

(51) Int. Cl. A61K 49/18; A61K 9/127

(52) U.S. Cl. CPC A61K 49/1812; A61K 9/127
USPC 424/9.321; 424/450; 435/375

(57) ABSTRACT

Novel liposomal nanoparticle that has been engineered to be particularly useful for the delivery of compounds to cells found in the peripheral nervous system, and to endothelial cells that form the blood brain barrier. These nanoparticles are intended to be useful for the delivery of compounds suitable for therapeutic purposes and imaging contrast agents that may not otherwise gain access to neuronal axons, or glial cells regions of the brain. Particularly advantageous for the purpose of targeting neural cells, endothelial cells of the blood vessels and epithelial cells of the choroid plexus that serve the brain is the inclusion, in the nanoparticles of cholesterol that surprisingly increases the affinity of the nanoparticles for such as Schwann cells, glial cells, and the like. Image contrast agents, such as those suitable for use in MRI techniques may also be delivered to neural cells, including of the peripheral nervous system. These advantageous liposomal nanoparticles at least comprise a phospholipid, a non-ionic surfactant, and cholesterol (or a derivative thereof).

### CROSS-REFERENCE TO RELATED APPLICATIONS

This application claims priority to U.S. Provisional Patent Application Ser. No. 61/620,600 entitled “NEUROPHILIC NANOPARTICLES” filed Apr. 5, 2012, and to U.S. Provisional Patent Application Ser. No. 61/697,492 entitled “NEUROPHILIC NANOPARTICLES” filed Sep. 6, 2012, the entireties of which are hereby incorporated by reference.

### TECHNICAL FIELD

The present disclosure is generally related to neurophilic liposomal nanoparticles useful as vehicles for the delivery of therapeutic, labeling, and imaging agents to neuronal and glial cells of the peripheral neural system. The present disclosure is further generally related to neurophilic liposomal nanoparticles useful as vehicles for the delivery of therapeutic, labeling, and therapeutic agents across the blood-brain barrier.

### BACKGROUND

Neurological disorders represent a large area of unmet medical need. This unmet need requires urgent attention particularly because the world's population is aging and the incidence of neurological disorders increases with age. Major neurological disorders can arise from neurons or glial cells within the central or the peripheral nervous system. A major challenge in battling neurological disorders is in delivering imaging, diagnostic and therapeutic reagents to the disease sites. For reagents to reach the brain, they must be able to cross the blood-brain barrier, which is lined with tight junctions that prevent macromolecules from entering into the brain. For reagents to reach motor or sensory neurons, they need to be able to travel the extensive lengths of the neuron projections.

Hereditary motor and sensory neuropathies (HMSN) and other neuromuscular disorders are common among the population, yet treatment options for affected individuals are limited. For example, currently there is no effective drug therapy for Charcot-Marie-Tooth (CMT) neuropathies and supportive care is limited to rehabilitation and surgical approaches. Delivery of therapeutic and diagnostic compounds to the neuromuscular system would provide invaluable tools to advance the care for affected individuals. Lipid nanoparticles are attractive options for delivery vehicles to the cells of the peripheral nervous system as they can be optimized for specific cell types, have good carrier capacity, and have low toxicity in vivo. In addition, both neurons and glial cells, such as Schwann cells, utilize the endocytic pathway, a mechanism by which certain nanoparticle formulations are taken up into cells. Liposomes can be administered locally or systemically, which offer options for targeting particular muscles of the body, such as the diaphragm, or the whole body using systemic administration through intravenous injection.

Clinical investigations of peripheral nerve lesions often involve nerve conduction studies and electromyography. Although these techniques allow the diagnosis of nerve damage and nerve conduction blocks, they cannot identify the underlying causes and location of nerve damage. Imaging methods such as Magnetic Resonance Imaging (MRI) can be used to supplement nerve conduction studies and electromyography as a non-invasive technology that provides good contrast for soft tissues. Gadolinium, an MRI contrast agent, allows changes in positive contrast to be more readily observable. However, this conventional MRI technique is not sufficiently sensitive in identifying early signs of neurodegeneration, or early signs of nerve regeneration from drug treatments (Bar-Or et al., (2011) CNS Drugs 25: 783-799; Bendszus et al., (2004) Exp. Neurol. 188: 171-177; Koltzenburg & Bendszus (2004) Curr. Opin. Neurol. 17: 621-626; Zivadinov R. (2007) Neurology 68: S72-S82). Also, much higher MRI resolution acquisition is needed in small animal models because the neurodegeneration in such models is typically less severe than in humans (Pirko & Johnson (2008) Curr. Top. Microbiol. Immunol. 318: 241-266). Thus, there is still an urgent need to develop higher MRI resolution acquisition for preclinical and clinical studies of peripheral nerve lesions.

### SUMMARY

Briefly described, one aspect of the present disclosure encompasses embodiments of a liposomal nanoparticle vehicle for the delivery of a compound to a neuronal or a glial cell, the vehicle comprising a phospholipid, a non-ionic surfactant, and cholesterol.

In the embodiments of this aspect of the disclosure, the phospholipid can be, but is not limited to, dioleoyl-phosphatidylcholine (DOPC), 1,2-dioleolyl-sn-glycero-3-phosphoethanolamine, or a combination thereof.

The non-ionic surfactant can be a block copolymer such as, for example, a tri-block copolymer. In some embodiments, the tri-block copolymer can have the formula (PEO)₇₅—(PPO)₃₀—(PEO)₇₅.

The liposomal nanoparticle vehicle of the disclosure can further comprise a compound desired to be delivered to a neuronal or a glial cell or to cross the blood-brain barrier.

In various embodiments, the compound can be a detectable label, a therapeutic agent, or an imaging agent. The detectable label can be, for example, a fluorescent label, a detectable metal-based label, a radiolabel, or an imaging agent that can provide or enhance imaging contrast when in a neuronal or a glial cell, or tissue of a recipient subject. In some embodiments of this aspect of the disclosure, the detectable can be gadolinium that can be bound to a chelating agent that may in turn be conjugated to a lipid component of the nanoparticle vehicle.

In some embodiments of this aspect of the disclosure, the imaging agent is gadolinium, or a derivatized variant thereof, such as, but not limited to Gadolinium-F (Gf) or Gadolinium-M.

In the embodiments of this aspect of the disclosure, the nanoparticle can have a diameter between about 100 nm to about 1000 nm, most preferably between about 50 nm to about 700 nm.

Another aspect of the disclosure encompasses embodiments of a method of imaging a neuronal cell or a glial cell, or a system of neuronal and glial cells, the method comprising delivering to a recipient animal or human subject a liposomal nanoparticle vehicle according to the disclosure and comprising an imaging agent, allowing the vehicle to deliver the imaging agent to a neuronal or a glial cell, and detect the label in the recipient neuronal cell or glial cell or in a system of neuronal and glial cells.

Another aspect of the disclosure encompasses embodiments of a method of diagnosing a neuropathological condition in an animal or human subject, the method comprising delivering to the animal or human subject a liposomal nanoparticle vehicle according to the disclosure, comprising a detectable label, allowing the vehicle to access peripheral neurons or glial cells of the recipient subject, detecting the label in the peripheral neural system of the subject, thereby determining the presence or absence of a neuromuscular pathology of the subject animal or human.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a series of digital images showing liposome uptake into normal non-myelinating Schwann cells. Non-myelinating Schwann cells after incubation with DOPC liposomes (200 μM) formulated with Tween 20 (Panel A), Tween 80 (Panel B), poloxamer 188 (P188) (Panel C), DOPC lipids only (Panel D), and no treatment (Panel E) were analyzed by fluorescence microscopy. After 8 h of uptake in 2% fetal calf serum-containing medium, a subset of cells displayed internalization of liposomes with poloxamer 188 (Panel C). Higher magnification of the boxed cells is shown in Panel C′. Nuclei were stained with Hoechst dye in blue. Scale bar represents 20 microns.

FIGS. 2A and 2B are digital images showing the uptake of fluorescent nanoparticles by motor neurons (FIG. 2A) and myelinating Schwann cells (FIG. 2B).

FIG. 3 is a graph showing the uptake of fluorescent liposomes by human SY5Y neurons and human brain microvascular endothelial cells (BMECs). SY5Y and BMECs were incubated with (200 NM) fluorescent liposomes having DOPC, DOPC/P188, DOPC/cholesterol, or DOPC/P188/cholesterol for 20 h at 37° C. Flow cytometry was performed to quantify liposome uptake.

FIG. 4 is a series of digital images showing that cholesterol improves liposome uptake into normal rat non-myelinating Schwann cells. DOPC liposomes (200 μM) formulated with P188 (Panel A), or P188/cholesterol (Panel B) or no treatment (Panel C) were incubated with normal Schwann cells and analyzed after 24 h of uptake in 2% fetal calf serum containing medium. Poloxamer-liposome uptake at 24 h is noticeably increased compared to that after 8 h (FIG. 4, Panel A, compared with FIG. 1, Panel C); the addition of cholesterol further enhances this effect (Panel B). Nuclei of the cells are stained with Hoechst dye.

FIG. 5 is a series of digital images showing green fluorescent liposomes are internalized by myelinating Schwann cells and sensory neurons. Dorsal root ganglion (DRG) explant cultures were established from normal mouse embryos and incubated with liposomes after two weeks of culture time (DIV14). DOPC/P188 (Panels A and D) or DOPC/P188/cholesterol (Panels B and E) liposomes were reconstituted in normal growth media to 200 μM and incubated for 24 hours. Internalization of DOPC/P188 liposomes in Schwann cells is low (Panel A) but enhanced with the addition of cholesterol (Panel B), whereas liposome uptake by DRG neurons is equally effective with DOPC/P188 (Panel D) or DOPC/P188/cholesterol (Panel E) formulations. Insets in Panels A and B represent enlarged images of Schwann cells marked with (*). Nuclei are stained with Hoechst dye in blue. Panels C and F are phase contrast images of regions from the peripheral regions of the explants that contain predominantly Schwann cells (Panel C), while clusters of neuronal cell bodies are localized more centrally (Panel F).

FIGS. 6A-6C illustrate that celastrol induces chaperone expression by Schwann cells. FIG. 6A: Total Schwann cell lysates (12 μg/lane) after treatment with free celastrol (Cel) for 16 h at the indicated doses were analyzed by western blotting for the levels of HSP70 and changes in LC3 II/I ratios. Geldanamycin (GA) or rapamcyin (RM), both at 50 nM, served as positive control for heat shock pathway or autophagy activation, respectively. Numbers below the blot indicate LC3-II to LC3-I ratio; FIG. 6B: Mouse DRG explant cultures, under myelinating conditions, were treated with celastrol dissolved in DMSO (0, 100 or 250 nM concentration) or with celastrol-filled liposomes (100 and 250 nM) for 96 h. Whole cell lysates (30 μg/lane) were analyzed for the levels of HSP70 and HSP27 proteins; FIG. 6C: DRGs depleted of Schwann cells treated with celastrol (250 nM and 500 nM) for 24 h were analyzed. GAPDH or UCHL served as loading controls. Molecular mass, in kDa.

FIG. 7 illustrates DOPC/188/Cholesterol liposomes were taken up by cortical neurons and glia. Single-plane confocal micrographs of cortical neurons (Panel A) and oligodendrocytes (Panel B) incubated with fluorescent DOPC/P188/Cholesterol liposomes (200 μM) for 24 h at 37° C. followed by a brief exposure to LysoTracker Red (1 μM, final 30 min) to label lysosomes. Cells loaded with Lysotracker only are shown in Panel C. Nuclei were stained with blue Hoescht dye. Scale bars, 10 μm.

FIGS. 8A and 8B illustrate that DOPC/P188/Cholesterol liposomes are taken up by human neural stem cells. Single-plane confocal micrographs of human neural stem cells incubated with fluorescent DOPC/P188/Cholesterol liposomes (300 μM) for 24 h at 37° C. (FIG. 8A). Untreated cells are shown in FIG. 8B. Nuclei, stained with DAPI dye, are shown. Scale bars, 10 μm.

FIGS. 9A and 9B illustrate that DOPC/188/Cholesterol liposomes are taken up by human SCA2 neuronal cells. Human neuronal cells specific for SCA2 disease were incubated with fluorescent DOPC/P188/Cholesterol liposomes (150 μM) for 24 h at 37° C. (FIG. 9A). Untreated cells are shown in FIG. 9B. Nuclei, stained with DAPI dye, are shown.

FIGS. 10A-10C illustrate that intravenous injection of DOPC/P188/Cholesterol liposomes led to their accumulation in brain choroid plexus epithelia cells and brain endothelial cells. Eight- to twelve-week old mice were injected intravenously via tail vein with 5 mg of DOPC/P188/Cholesterol liposomes containing 0.5 mg Cy5 fluorescent dye reconstituted in 150 μl sterile saline (0.9% NaCl). Mice were euthanized by CO₂ asphyxiation 4-24 h later. Brain tissues were obtained, fixed in 4% paraformaldehyde-phosphate buffered saline (PBS) for 3 h at room temperature, followed by cryoprotection in 30% sucrose-PBS overnight at 4° C. Cryosections (20 μm thickness) were collected onto Superfrost plus slides, rinsed twice with PBS, and stained with Hoescht dye (Invitrogen). Representative images at 4 h (FIG. 10A, shown at higher magnification in inset A′) and 24 h (FIG. 10B) post-injection. Non-injected brain is shown in FIG. 10C.

FIG. 11 shows T1-weighted MR images and measurements of Gf-filled liposomes. Samples: 1: Gf standard (1.0 mM), T1 (ms) 129, T2 (ms) 49; 2: Liposomal Gf (0.0001 mM), T1 (ms) 2685, T2 (ms) 210; 3: Liposomal Gf (0.001 mM), T1 (ms) 2604, T2 (ms) 217; 4: Liposomal Gf (0.01 mM), T1 (ms) 2126, T2 (ms) 210; 5: Liposomal Gf (0.1 mM), T1 (ms) 727, T2 (ms) 155; 6: Liposomal Gf (1.0 mM), T1 (ms) 88, T2 (ms) 40; 7: Empty liposomes (equivalent to 1 mM; liposomal Gf concentration), T1 (ms) 2360, T2 (ms) 224; 8: Saline, T1 (ms) 2655, T2 (ms) 213.

FIG. 12 illustrates in vivo liposome clearance. Two-month-old C57/BI6 mice were injected intravenously with Cy5-labeled DOPC/P188/Cholesterol liposomes (20 mM; 0.2 ml). Mice were sacrificed 4 and 24 h post-injection. Mouse tissues, such as the liver (Panels A-C), kidney (Panels D-F) and large intestines (Panels G-I) were removed, processed and examined for liposome fluorescence. At 4 h, liposomes were present at high levels in both the liver hepatocytes (Panel A, and enlarged inset) and kidney (Panel D) but much less in the large intestines (Panel G). After 24 hours, liposome detection was overall reduced in the liver (Panel B; enlarged inset shows bile canniculi) and kidney (Panel E), and nearly undetectable in the large intestine (Panel H). Non-injected liver, kidney and large intestine tissues are shown in Panels C, F, and I respectively. Nuclei are stained with Hoescht dye. Scale bars, 100 microns, and 10 microns in insets (Panels A and B).

FIGS. 13A and 13B illustrate that DOPC/P188/Cholesterol liposomes accumulated in peripheral nerves after intravenous or intramuscular injection. Eight-week old mice were injected intravenously via tail vein with 5 mg of DOPC/P188/Cholesterol liposomes containing 0.5 mg CF fluorescent dye reconstituted in 150 μl sterile 0.9% saline (FIG. 13A). Eight-week old mice were injected intramuscularly with 10 μl of 20 mM DOPC liposomes labeled with CF fluorescent dye (FIG. 13B). Mice were euthanized by CO₂ asphyxiation 4 or 24 h later. Peripheral nerves were obtained, fixed in 4% paraformaldehyde-phosphate buffered saline (PBS) for 3 h at room temperature, followed by cryoprotection in 30% sucrose-PBS overnight at 4° C. Cryosections (20 μm thickness) were collected onto Superfrost plus slides, rinsed twice with PBS, and stained with Hoescht dye (Invitrogen). Representative peripheral nerve images 4 h post-intravenous injection (FIG. 13A) and 4 h post-intramuscular injection (FIG. 10B). Non-injected nerve is shown as inset in FIG. 13A, inset A′.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein.

Prior to describing the various embodiments, the following definitions are provided and should be used unless otherwise indicated.

### DEFINITIONS

The term “formulation” as used herein refers to a composition that may be a stock solution of the components, or a composition, preferably including a dilutant such as water or other pharmaceutically acceptable carrier that may be available for distribution including to a patient or physician.

The term “detectable moiety” as used herein refers to a label molecule (isotopic or non-isotopic) which is incorporated indirectly or directly into a liposomal nanoparticle according to the disclosure, wherein the label molecule facilitates the detection of the nanoparticle in which it is incorporated. Thus, “detectable moiety” is used synonymously with “label molecule”. Label molecules, known to those skilled in the art as being useful for detection, include chemiluminescent or fluorescent molecules. Various fluorescent molecules are known in the art which are suitable for use to label a nucleic acid for the method of the present invention. The protocol for such incorporation may vary depending upon the fluorescent molecule used. Such protocols are known in the art for the respective fluorescent molecule.

The term “label” or “tag” as used herein refers to a molecule that, when appended by, for example, without limitation, covalent bonding or hybridization to another moiety, for example, also without limitation, a nanoparticle provides or enhances a means of detecting the other moiety. A fluorescence or fluorescent label or tag emits detectable light at a particular wavelength when excited at a different wavelength. A radiolabel or radioactive tag emits radioactive particles detectable with an instrument such as, without limitation, a scintillation counter. Other signal-generation detection methods include: chemiluminescence, electrochemiluminescence, raman, colorimetric, hybridization protection assay, and mass spectrometry. Radionuclides may be either therapeutic or diagnostic; diagnostic imaging using such nuclides is also well known. Typical diagnostic radionuclides include, but are not limited to, ⁹⁹Tc, ⁹⁵Tc, ¹¹¹In, ⁶²Cu, ⁶⁴Cu, ⁶⁷Ga, ⁶⁸Ga.

The term “contrast agent” as used herein refers to an agent that when delivered to an animal or human subject can improve the image obtained by a method such as magnetic resonance imaging (MRI). Such agents may include, but are not limited to gadolinium, iron oxide, manganese and magnesium salts, and the like that may be formulated into pharmaceutically acceptable compositions for administering in vivo with limited and acceptable degrees of undesirable side effects. One suitable MRI contrast agent for incorporation into the liposomal nanoparticle delivery vehicles of the disclosure is gadolinium (Gd), and derivatized variants thereof. A particularly useful such derivative, but not intended to be limiting, is Gadofluorine (GdF, Bayer Schering Pharma AG), a gadolinium analogue that is an amphiphilic, macrocyclic, gadolinium-containing complex. It is a derivative of Gd-DO3A containing a perfluorooctyl side chain and a mannose moiety. Other Gd derivatives for use as an MRI contrast agent are, but not limited to, Carbocyanine-labelled GdF (cc-GdF), Gd-DTPA (MAGNEVIST® Bayer Schering Pharma, Berlin, Germany), Gd-DO3A and the like.

The term “imaging agent” as used herein refers to a labeling moiety that is useful for providing an indication of the position of the label and adherents thereto, in a cell or tissue of an animal or human subject, or a cell or tissue under in vitro conditions. Such agents may include those that provide detectable signals such as fluorescence, luminescence, radioactivity, or can be detected by such methods as MRI imaging, PET imaging and the like.

Chelating agents containing paramagnetic metals for use in magnetic resonance imaging can also be employed as ancillary agents. Typically, a chelating agent containing a paramagnetic metal is associated with a coating on the nanoparticles. The chelating agent can be coupled directly to one or more of components of the nanoparticle such as functional amino groups. Suitable chelating agents include, but are not limited to, a variety of multi-dentate compounds including EDTA, DPTA, DOTA, and the like.

It will be understood by one of ordinary skill in the art that when referring to a population of nanoparticles as being of a particular “size”, what is meant is that the population is made up of a distribution of sizes around the stated “size”. Unless otherwise stated, the “size” used to describe a particular population of nanoparticles will be the mode of the size distribution (i.e., the peak size). By reference to the “size” of a nanoparticle is meant the length of the largest straight dimension of the nanoparticle. For example, the size of a perfectly spherical nanoparticle is its diameter.

The term “nanoparticle” As used herein refers to a particle having a diameter of between about 1 and about 1000 nm, preferably between about 100 nm and 1000 nm, and most preferably between about 50 nm and 700 nm. Similarly, by the term “nanoparticles” is meant a plurality of particles having an average diameter of between about 50 and about 1000 nm.

The following is in an exemplary list of amphiphilic random and alternating copolymers useful in the nanoparticles of the disclosure: random copolymer poly(dimethyl siloxane-co-diphenyl siloxane); random copolymer poly(methyl acrylate-co-acrylic acid); random copolymer poly(methyl methacrylate-co-n-butyl acrylate); random copolymer poly(methyl methacrylate-co-t-butyl acrylate); random copolymer poly(methyl methacrylate-co-hydroxypropyl acrylate); random copolymer poly(tetrahydrofuranyl methacrylate-co-ethyl methacrylate); random copolymer poly(styrene-co-4-bromostyrene); random copolymer poly(styrene-co-butadiene); random copolymer poly(styrene-co-diphenyl ethylene); random copolymer poly(styrene-co-t-butyl methacrylate); random copolymer poly(styrene-co-t-butyl-4-vinyl benzoate); random copolymer poly(styrene-co-p-carboxyl chloro styrene); random copolymer poly(styrene-co-p-chloromethyl styrene); random copolymer poly(styrene-co-methyl methacrylate); random copolymer poly(styrene-co-4-hydroxystyrene); random copolymer poly(styrene-co-4-vinyl benzoic acid); random copolymer poly(styrene-co-4-vinyl pyridine); alternating copolymer poly(carbo tert.butoxy α-methyl styrene-alt-maleic anhydride); alternating copolymer poly(carbo tert.butoxy norbornene-alt-maleic anhydride); alternating copolymer poly(α-methyl styrene-alt-methyl methacrylate); and alternating copolymer poly(styrene-alt-methyl methacrylate).

The following in an exemplary list of amphiphilic copolymers: poly((meth)acrylic acid) based copolymers (e.g., poly(acrylic acid-β-methyl methacrylate); poly(methyl methacrylate-β-acrylic acid); poly(methyl methacrylate-β-sodium acrylate); poly(sodium acrylate-β-methyl methacrylate); poly(methacrylic acid-β-neopentyl methacrylate); poly(neopentyl methacrylate-β-methacrylic acid); poly(t-butyl methacrylate-β-ethylene oxide); poly(methyl methacrylate-β-sodium methacrylate); and poly(methyl methacrylate-β-N,N-dimethyl acrylamide)), polydiene and hydrogenated polydiene based copolymers (e.g., poly(butadiene(1,2 addition)-β-methylacrylic acid; poly(butadiene(1,4 addition)-β-acrylic acid); poly(butadiene(1,4 addition)-β-sodium acrylate); poly(butadiene(1,4 addition)-β-ethylene oxide; poly(butadiene(1,2 addition)-β-ethylene oxide); poly(butadiene(1,2 addition)-β-ethylene oxide)-hydroxy benzoic ester terminal group; 4-methoxy benzyolester terminated poly(butadiene-β-ethylene oxide) diblock copolymer; poly(butadiene-β-N-methyl 4-vinyl pyridinium iodide); poly(isoprene-β-N-methyl 2-vinyl pyridinium iodide); poly(isoprene-β-ethylene oxide) (1,4 addition); poly(isoprene-β-ethylene oxide) (1,2 and 3,4 addition); poly(propylene-ethylene-β-ethylene oxide); and hydrogonated poly(isoprene-β-ethylene oxide) (1,2 addition)), hydrogentated diene based copolymers (e.g., poly(ethylene-β-ethylene oxide) and poly(isoprene-(3-ethylene oxide)), poly(ethylene oxide) based copolymers (e.g., poly(ethylene oxide-β-acrylic acid); poly(ethylene oxide-β-ε-caprolactone); poly(ethylene oxide-β-(4′-cyanobiphenyl-4-yloxy)hexyl methacrylate); poly(ethylene oxide-β-lactide); poly(ethylene oxide-β-2-hydroxyethyl methacrylate); poly(ethylene oxide-β-methyl methacrylate); poly(-methyl methacrylate-β-ethylene oxide); poly(ethylene oxide-β-methacrylic acid); poly(ethylene oxide-β-2-methyl oxazoline); poly(ethylene oxide-β-propylene oxide); poly(ethylene oxide-β-t-butyl acrylate); poly(ethylene oxide-β-tetrahydrofurfuryl methacrylate); and poly(ethylene oxide-β-N,N-dimethylethylmethacrylate)), polyisobutylene based copolymers (e.g., poly(isobutylene-β-ethylene oxide)), polystyrene based copolymers (e.g., poly(styrene-β-acrylic acid); poly(styrene-β-sodium acrylate); poly(styrene-β-acrylamide); poly(p-chloromethyl styrene-β-acrylamide); poly(styrene-co-β-chloromethyl styrene-β-acrylamide); poly(styrene-co-β-chloromethyl styrene-β-acrylic acid); poly(styrene-β-cesium acrylate); poly(styrene-β-ethylene oxide); poly(4-styrenesulfonic acid-β-ethylene oxide); poly(styrene-β-methacrylic acid); poly(styrene-β-sodium methacrylate); poly(styrene-β-N-methyl 2-vinyl pyridinium iodide); and poly(styrene-β-N-methyl-4-vinyl pyridinium iodide)), polysiloxane based copolymers (e.g., poly(dimethylsiloxane-β-acrylic acid)), poly(2-vinyl naphthalene) based copolymers (e.g., poly(2-vinyl naphthalene-β-3-acrylic acid)), poly(vinyl pyridine and N-methyl vinyl pyridinium iodide) based copolymers (e.g., poly(2-vinyl pyridine-β-ethylene oxide); poly(N-methyl 2-vinyl pyridinium iodide-β-ethylene oxide); and poly(N-methyl 4-vinyl pyridinium iodide-β-methyl methacrylate)).

The following in an exemplary list of amphiphilic diblock copolymers: poly(meth)acrylate based copolymers (e.g., poly(n-butyl acrylate-β-methyl methacrylate); poly(n-butyl acrylate-β-dimethylsiloxane-co-diphenyl siloxane); poly(t-butyl acrylate-β-methyl methacrylate); poly(t-butyl acrylate-β-4-vinylpyridine); poly(2-ethyl hexyl acrylate-β-4-vinyl pyridine); poly(t-butyl methacrylate-β-2-vinyl pyridine); poly(2-hydroxyl ethyl acrylate-β-neopentyl acrylate); poly(2-hydroxylethyl methacrylate-β-neopentyl methacrylate); poly(2-hydroxylethyl methacrylate-β-n-butyl methacrylate); poly(2-hydroxyl ethyl methacrylate-β-t-butyl methacrylate); poly(methyl methacrylate-β-acrylonitrile); poly(methyl methacrylate-β-t-butyl methacrylate); poly(isotactic methyl methacrylate-β-syndiotactic methyl methacrylate); poly(methyl methacrylate-β-t-butyl acrylate); poly(methyl methacrylate-β-trifluoroethyl methacrylate); poly(methyl methacrylate-β-2-hydroxyethyl methacrylate with cholesteryl chloroformate); poly(methyl methacrylate-β-disperse red 1 acrylate); poly(methyl methacrylate-β-2-hydroxyethyl methacrylate); poly(methyl methacrylate-β-neopentyl acrylate); and poly(methacrylate-β-2-pyranoxy ethyl methacrylate)), polydiene based copolymers (e.g., poly(butadiene(1,2 addition)-β-1-butyl methacrylate); poly(butadiene(1,2 addition)-β-s-butyl methacrylate); poly(butadiene(1,4 addition)-β-t-butyl acrylate; poly(butadiene(1,2 addition)-β-t-butyl acrylate; poly(butadiene(1,2 addition)-β-t-butyl methacrylate); poly(butadiene(1,4 addition)-β-ε-caprolactone); poly(butadiene((1,4 addition)-β-dimethylsiloxane); poly(butadiene(1,4 addition)-β-methyl methacrylate) (syndiotactic); poly(butadiene(1,2 addition)-β-methyl methacrylate); poly(butadiene(1,4 addition)-β-4-vinyl pyridine; poly(isoprene(1,4 addition)-β-methyl methacrylate(syndiotactic)); poly(isoprene(1,4 addition)-β-2-vinyl pyridine; poly(isoprene(1,2 addition)-β-4-vinyl pyridine); and poly(isoprene(1,4 addition)-β-4-vinyl pyridine)), polyisobutylene based copolymers (e.g., poly(isobutylene-β-t-butyl methacrylate); poly(isobutylene-β-ε-caprolactone); poly(isobutylene-β-dimethylsiloxane); poly(isobutylene-β-methyl methacrylate); poly(isobutylene-β-4-vinyl pyridine), polystyrene based copolymers (e.g., poly(styrene-β-n-butyl acrylate); poly(styrene-β-t-butyl acrylate); poly(styrene-β-t-butyl acrylate), broad distribution; poly(styrene-β-disperse red 1 acrylate); poly(p-chloromethyl styrene-β-t-butyl acrylate); poly(styrene-β-N-isopropyl acrylamide); poly(styrene-β-n-butyl methacrylate); poly(styrene-β-t-butyl methacrylate); poly(styrene-(3-cyclohexyl methacrylate); poly(styrene-β-cholesteryloxycarbonyloxy ethyl methacrylate); poly(styrene-β-N,N-dimethyl amino ethyl methacrylate); poly(styrene-β-ethyl methacrylate); poly(styrene-β-2-hydroxyethyl methacrylate); poly(styrene-β-2-hydroxypropyl methacrylate); poly(styrene-β-methyl methacrylate); poly(styrene-β-methylmethacrylate); poly(styrene-β-n-propyl methacrylate); poly(styrene-β-butadiene(1,4 addition)); poly(styrene-β-butadiene(1,2 addition)); poly(styrene-β-isoprene(1,4 addition)); poly(styrene-β-isoprene(1,2 addition or 3,4 addition)); poly(styrene-β-isoprene(1,4 addition)), hydrogenated; tapered block copolymer poly(styrene-(3-butadiene); tapered block copolymer poly(styrene-β-ethylene); poly(styrene-β-ε-caprolactone); poly(styrene-β-l-lactide); poly(styrene-β-dimethylsiloxane), trimethylsilane endgroup; poly(styrene-β-dimethylsiloxane), silanol endgroup; poly(styrene-Vmethyl phenyl siloxane); poly(styrene-β-ferrocenyldimethylsilane); poly(styrene-β-t-butyl styrene); poly(styrene-β-t-butoxystyrene); poly(styrene-β-4-hydroxyl styrene); poly(4-amino benzyl-β-styrene); poly(styrene-β-2-vinyl pyridine); poly(styrene-β-4-vinyl pyridine); and poly(α-methylstyrene-β-4-vinyl pyridine), poly(vinyl naphthalene) based copolymers (e.g., poly(2-vinyl naphthalene-β-n-butyl acrylate), poly(2-vinyl naphthalene-β-t-butyl acrylate); poly(2-vinyl naphthalene-β-methyl methacrylate); and poly(2-vinyl naphthalene-β-dimethylsiloxane)), poly(vinyl pyridine) based copolymers (e.g., poly(2-vinyl pyridine-β-ε-caprolactone); poly(2-vinyl pyridine-β-methyl methacrylate); and poly(4-vinyl pyridine-β-methyl methacrylate)), poly(propylene oxide-β-ε-caprolactone) (e.g., poly(propylene oxide-β-ε-caprolactone)), polysiloxane based copolymers (e.g., poly(dimethylsiloxane-β-n-butyl acrylate); poly(dimethylsiloxane-β-t-butyl acrylate); poly(dimethylsiloxane-β-t-butyl methacrylate); poly(dimethylsiloxane-β-ε-caprolactone); poly(dimethylsiloxane-β-6-(4′-cyanobiphenyl-4-yloxy)hexyl methacrylate); poly(dimethylsiloxane-β-1-ethoxy ethyl methacrylate); poly(dimethylsiloxane-β-hydroxy ethyl acrylate); and poly(dimethylsiloxane-β-methyl methacrylate)), adipic anhydride based copolymers (e.g., poly(ethylene oxide-β-adipic anhydride); poly(propylene oxide-β-adipic anhydride); poly(dimethyl siloxane-β-adipic anhydride); poly(methyl methacrylate-β-adipic anhydride); and poly(2-vinyl pyridine-β-adipic anhydride)).

The following in an exemplary list of amphiphilic a-β-a triblock copolymers: poly((meth)acrylate) based triblock copolymers (e.g., poly(n-butyl acrylate-β-9,9-di-n-hexyl-2,7-fluorene-β-n-butyl acrylate); poly(t-butyl acrylate-β-9,9-di-n-hexyl-2,7-fluorene-β-t-butyl acrylate); poly(acrylic acid-β-9,9-di-n-hexyl-2,7-fluorene-β-acrylic acid); poly(t-butyl acrylate-β-methyl methacrylate-β-t-butyl acrylate); poly(t-butyl acrylate-β-styrene-β-t-butyl acrylate); poly(methyl methacrylate-β-butadiene(1,4 addition)-β-methyl methacrylate); poly(methyl methacrylate-β-n-butyl acrylate-β-methyl methacrylate); poly(methyl methacrylate-β-t-butyl acrylate-β-methyl methacrylate); poly(methyl methacrylate-β-t-butyl methacrylate acid-β-methyl methacrylate); poly(methyl methacrylate-β-methacrylic acid-β-methyl methacrylate); poly(methyl methacrylate-β-dimethylsiloxane-β-methyl methacrylate); poly(methyl methacrylate-β-9,9-di-n-hexyl-2,7-fluorene-β-methyl methacrylate); poly(methyl methacrylate-β-styrene-β-methyl methacrylate); poly(trimethylamonium iodide ethyl methacrylate-β-9,9-di-n-hexyl-2,7-fluorene-β-trimethylamonium iodide ethyl methacrylate); poly(N,N-dimethyl amino ethyl methacrylate-β-9,9-di-n-hexyl-2,7-fluorene-β-N,N-dimethyl amino ethyl methacrylate); and poly(N,N-dimethyl amino ethyl methacrylate-β-3-propylene oxide-β-N,N-dimethyl amino ethyl methacrylate)), polybutadiene based triblock copolymers (e.g., poly(butadiene(1,4 addition)-β-styrene-β-butadiene(1,4 addition))), poly(oxirane) based triblock copolymers (e.g., poly(ethylene oxide-β-9,9-di-n-hexyl-2,7-fluorene-β-ethylene oxide); poly(ethylene oxide-β-propylene oxide-β-ethylene oxide); poly(ethylene oxide-β-3-styrene-β-3-ethylene oxide); and poly(propylene oxide-β-dimethyl siloxane-β-propylene oxide)), polylactone and polylactide diblock copolymers (e.g., poly(lactide-β-3-ethylene oxide-β-lactide); poly(caprolactone-β-ethylene oxide-β-caprolactone); and alpha,-ωdiacrylonyl terminated poly(lactide-β-ethylene oxide-β-lactide)), polyoxazoline based triblock copolymers (e.g., poly(2-methyl oxazoline-β-dimethyl siloxane-β-2-methyl oxazoline))), polystyrene based triblock copolymers (e.g., poly(styrene-β-acrylic acid-β-3-styrene); poly(styrene-β-butadiene (1,4 addition)-β-styrene); poly(styrene-β-butadiene (1,2 addition)-β-styrene); poly(styrene-β-butylene-β-styrene); poly(styrene-β-n-butyl acrylate-β-styrene); poly(styrene-β-t-butyl acrylate-β-styrene); poly(styrene-β-9,9-di-n-hexyl-2,7-fluorene-β-styrene); poly(styrene-β-ethyl acrylate-β-styrene); poly(styrene-β-isoprene-β-styrene); poly(styrene-β-ethylene oxide-β-styrene); poly(styrene-β-4-vinyl pyridine-β-styrene); and poly(styrene-β-dimethyl siloxane-(3-styrene)), poly(vinyl pyridine) based triblock copolymers (e.g., poly(2-vinyl pyridine-β-butadiene(1,2 addition)-β-2-vinyl pyridine); poly(2-vinyl pyridine-β-styrene-β-2-vinyl pyridine); and poly(4-vinyl pyridine-β-styrene-β-4-vinyl pyridine).

The following in an exemplary list of amphiphilic a-β-c triblock copolymers: poly(styrene-β-butadiene-β-methyl methacrylate) (e.g., poly(styrene-β-butadiene-β-methyl methacrylate)), poly(styrene-β-butadiene-β-2-vinyl pyridine) (e.g., poly(styrene-β-butadiene-β-2-vinyl pyridine)), poly(styrene-β-t-butyl acrylate-β-methyl methacrylate) (e.g., poly(styrene-β-t-butyl acrylate-β-methyl methacrylate)), poly(styrene-β-isoprene-β-glycidyl methacrylate) (e.g., poly(styrene-β-isoprene-β-glycidyl methacrylate)), poly(styrene-β-2-vinyl pyridine-β-ethylene oxide) (e.g., poly(styrene-β-2-vinyl pyridine-β-ethylene oxide)), poly(styrene-β-anthracene methyl methacrylate-β-methymethacrylate) (e.g., poly(styrene-β-anthracene methyl methacrylate-β-methymethacrylate)), poly(styrene-β-t-butyl acrylate-β-2-vinyl pyridine) (e.g., poly(styrene-β-t-butyl acrylate-β-2-vinyl pyridine)), and poly(styrene-β-t-butyl methacrylate-β-2-vinyl pyridine) (e.g., poly(styrene-β-t-butyl methacrylate-β-2-vinyl pyridine)).

The following in an exemplary list of amphiphilic functionalized diblock and triblock copolymers: amino terminated poly(dimethylsiloxane-β-diphenylsiloxane); amino terminated poly(styrene-β-isoprene); amino terminated poly(ethylene oxide-β-lactone); hydroxy terminated poly(styrene-O-2-vinyl pyridine); hydroxy terminated polystyrene-β-poly(methyl methacrylate); α-hydroxy terminated poly(styrene-β-butadiene(1,2-addition)); 4-methoxy benzyolester terminated poly(butadiene-β-ethylene oxide) diblock copolymer; succinic acid terminated poly(butadiene-β-ethylene oxide) diblock copolymer; α,ω-disuccinimidyl succinate terminated poly(ethylene oxide-propylene oxide-ethylene oxide); cabinol at the junction of poly(styrene-β-isoprene(1,4 addition)); and silane at the junction of poly(styrene-β-2-vinyl pyridine).

In addition, the following is an exemplary list of amphiphilic block copolymers: poly(1-vinylpyrrolidone-co-vinyl acetate); poly(ethylene-co-propylene-co-5-methylene-2-norbornene); poly(styrene-co-acrylonitrile); poly(2-vinylpyridine-co-styrene); poly(ethylene-co-methacrylic acid) sodium salt; poly(acrylonitrile-co-butadiene-co-styrene); poly(vinyl chloride-co-vinyl acetate-co-maleic acid); poly(ethylene-co-vinyl acetate); poly(ethylene-co-ethyl acrylate); poly(4-vinylpyridine-co-styrene); poly(vinyl butyral-co-vinyl alcohol-co-vinyl acetate); poly(methyl methacrylate co-methacrylic acid); poly-(vinyl chloride-co-vinyl acetate-co-hydroxypropyl acrylate); Luviquat® HM 552; poly(vinyl chloride-co-vinyl acetate-co-vinyl alcohol); poly(styrene-co-divinylbenzene); poly(DL-lactide-co-glycolide); poly(acrylonitrile-co-methyl acrylate); poly[(vinyl chloride-co-(1-methyl-4-vinylpiperazine)]; poly(2-isopropenyl-2-oxazoline-co-methyl methacrylate); poly(tetrafluoroethylene oxide-co-difluoromethylene oxide) α,ω-diol, ethoxylated; poly[dimethylsiloxane-co-methyl(3-hydroxypropyl)siloxane]-graft-poly(ethylene glycol) methyl ether; poly(acrylonitrile-co-methacrylonitrile); poly(ethylene-co-1-butene); poly(vinylidene fluoride co-hexafluoropropylene); poly(ethylene-co-1-octene); poly(ethylene-co-methyl acrylate); poly(acrylonitrile-co-butadiene), amine terminated; poly(perfluoropropylene oxide-co-perfluoroformaldehyde); poly(butyl methacrylate-co-isobutyl methacrylate); poly(styrene-co-maleic anhydride), partial isooctyl ester, cumene terminated; poly(acrylonitrile-co-butadiene-co-acrylic acid), dicarboxy terminated; poly(vinyl alcohol-co-ethylene); poly(dimethylsiloxane-co-methylphenylsiloxane); poly(styrene-co-maleic anhydride); poly(Bisphenol A-co-epichlorohydrin); poly(styrene-co-butadiene); poly[(R)-3-hydroxybutyric acid-co-(R)-3-hydroxyvaleric acid]; poly(vinyl alcohol-co-vinyl acetate-co-itaconic acid); poly(methylstyrene-co-indene), hydrogenated; poly(4-vinylphenol-co-2-hydroxyethyl methacrylate); poly(styrene-co-maleic anhydride), cumene terminated; poly(methyl methacrylate-co-ethylene glycol dimethacrylate); poly(ethylene-co-propylene); poly(styrene-co-maleic acid), partial isobutyl/methyl mixed ester; poly(Bisphenol A-co-epichlorohydrin), glycidyl end-capped; poly(methyl methacrylate-co-methacrylic acid); poly(2-acrylamido-2-methyl-1-propanesulfonic acid-co-acrylonitrile); poly(propylene-co-tetrafluoroethylene); poly(butyl methacrylate-co-methyl methacrylate); poly(dimethylsiloxane-co-alkylmethylsiloxane); poly(acrylic acid-co-acrylamide) potassium salt; poly(oxymethylene-co-1,3-dioxepane); poly(chlorotrifluoroethylene-co-vinylidene fluoride); poly(melamine-co-formaldehyde), acrylated solution; poly(pentafluorostyrene-co-glycidyl methacrylate); poly(1,1,1,3,3,3-hexafluoroisopropyl methacrylate-co-glycidyl methacrylate); poly(2,2,3,4,4,4,-hexafluorobutyl methacrylate-co-glycidyl methacrylate); poly(2,2,3,3,3-pentafluoropropyl methacrylate-co-glycidyl methacrylate); poly[(propylmethacryl-heptaisobutyl-PSS)-co-(n-butylmethacrylate)]; poly(pyromellitic dianhydride-co-4,4′-oxydianiline), amic acid solution; poly(tert-butyl methacrylate-co-glycidyl methacrylate); poly[(propylmethacryl-heptaisobutyl-PSS)-co-hydroxyethylmethacrylate]; poly[(m-phenylenevinylene)-co-(2,5-dioctoxy-β-phenylenevinylene)]; poly[(methylmethacrylate)-co-(9-anthracenylmethyl methacrylate)]; poly[(methylmethacrylate)-co-(2-naphthylacrylate)]; poly[methylmethacrylate-co-(7-(4-trifluoromethyl)coumarin methacrylamide)]; poly[(methylmethacrylate)-co-(9-anthracenylmethyl acrylate)]; poly[(methylmethacrylate)-co-(9H-carbazole-9-ethylmethacrylate)]; poly[(propylmethacryl-heptaisobutyl-PSS)-co-(methylmethacrylate)]; poly[(isobutylene-alt-maleic acid), ammonium salt)-co-(isobutylene-alt-maleic anhydride)]; poly(ethylenecarbonyl-co-propylenecarbonyl); poly[4,5-difluoro-2,2-bis(trifluoromethyl)-1,3-dioxole-co-tetrafluoroethylene]; poly(dimethylsiloxane-co-diphenylsiloxane), trimethylsilyl terminated; poly(dimethylsiloxane-co-methylhydrosiloxane), trimethylsilyl terminated; poly(dimethylsiloxane-co-diphenylsiloxane), divinyl terminated; poly(styrene-co-methyl methacrylate); poly(styrene-co-α-methylstyrene); poly(1,4-cyclohexanedimethylene terephthalate-co-ethylene terephthalate); AMBERJET® 4200; poly[dimethylsiloxane-co-methyl(3-hydroxypropyl)siloxane]-graft-poly(ethylene glycol) [3-(trimethylammonio)propyl chloride]ether solution; poly[dimethylsiloxane-co-methyl(3-hydroxypropyl)siloxane]-graft-poly(ethylene/propylene glycol); poly(ethylene-co-butyl acrylate); poly(ethylene-co-ethyl acrylate-co-maleic anhydride); poly(ethyl methacrylate-co-methyl acrylate); poly(ethylene-co-1-butene-co-1-hexene); poly(melamine-co-formaldehyde), isobutylated solution; poly[Bisphenol A carbonate-co-4,4′-(3,3,5-trimethylcyclohexylidene)diphenol carbonate]; poly(acrylamide-co-acrylic acid); poly(styrene-co-maleic acid), partial sec-butyl/methyl mixed ester; poly(4-hydroxybenzoic acid-co-6-hydroxy-2-naphthoic acid); poly[butylene terephthalate-co-poly(alkylene glycol) terephthalate]; poly(ethylene-co-vinyl acetate-co-methacrylic acid); poly(melamine-co-formaldehyde), methylated; poly(acrylonitrile-co-butadiene), dicarboxy terminated; poly(vinyl chloride-co-vinyl acetate-co-2-hydroxypropyl acrylate); poly(tetrafluoroethylene oxide-co-difluoromethylene oxide) α,ω-diol; poly(melamine-co-formaldehyde), butylated solution; poly[(phenyl glycidyl ether)-co-formaldehyde]; poly(acrylamide-co-diallyldimethylammonium chloride) solution; poly(tetrafluoroethylene-co-perfluoro(propylvinyl ether)); poly(4-vinylpyridine-co-butyl methacrylate); poly(dimer acid-co-alkyl polyamine); poly(1-vinylpyrrolidone-co-2-dimethylaminoethyl methacrylate), quaternized solution; poly(methyl methacrylate-co-ethyl acrylate); Luviquat®FC 550; poly(vinyltoluene-co-α-methylstyrene); poly(epichlorohydrin-co-ethylene oxide-co-allyl glycidyl ether); poly(dimethylsiloxane-co-methylhydrosiloxane); polybutadiene-graft-poly(methyl acrylate-co-acrylonitrile); poly(styrene-co-maleic anhydride), partial 2-butoxyethyl ester, cumene terminated; poly(dimethylamine-co-epichlorohydrin) solution; poly(ethylene-co-acrylic acid); poly(acrylamide-co-acrylic acid) partial sodium salt; poly(hexafluoropropylene oxide-co-difluoromethylene oxide)monoalkylamide; poly(1-vinylpyrrolidone-co-2-dimethylaminoethyl methacrylate) solution; poly(acrylic acid-co-maleic acid) sodium salt; poly(ethylene-co-acrylic acid, zinc salt); poly(ethylene-co-tetrafluoroethylene); poly(2,2,2-trifluoroethyl methacrylate-co-glycidyl methacrylate); poly(pentabromophenyl acrylate-co-glycidyl methacrylate); poly(2,2,3,3,4,4,4-heptafluorobutyl methacrylate-co-glycidyl methacrylate; poly[methylmethacrylate-co-(disperse yellow 7 methacrylate)]; poly(2,2,3,3-tetrafluoropropyl methacrylate-co-glycidyl methacrylate); poly(pentabromophenyl methacrylate-co-glycidylmethacrylate); poly[methylmethacrylate-co-(Disperse Orange 3 methacrylamide)]; poly[((S)-( [symbol] )(4-nitrophenyl)-2-pyrrolidinemethyl)acrylate-co-methylmethacrylate]; poly[(methylmethacrylate)-co-(Disperse Red 13 methacrylate)]; poly[(methylmethacrylate)-co-(Disperse Red 13 acrylate)]; poly[methylmethacrylate-co-(N-(1-naphthyl)-N-phenylacrylamide)]; poly[(propylmethacryl-heptaisobutyl-PSS)-co-styrene]; poly(pyromellitic dianhydride-co-thionin); poly(ethylene glycol)-co-4-benzyloxybenzyl alcohol, polymer-bound; poly[(isobutylene-alt-maleimide)-co-(isobutylene-alt-maleic anhydride)]; poly[dimethylsiloxane-co-(3-aminopropyl)methylsiloxane]; poly[dimethylsiloxane-co-β-(2-(2-hydroxyethoxy)ethoxy)propyl)methylsiloxane]; poly(vinylidene chloride-co-acrylonitrile-co-methyl methacrylate); poly(ethylene-co-1,2-butylene)diol; poly(DL-lactide-co-caprolactone) (40:60); poly(methyl methacrylate-co-butyl methacrylate); poly(tetrafluoroethylene oxide-co-difluoromethylene oxide)α,ω-diol bis(2,3-dihydroxypropyl ether); poly[dimethylsiloxane-co-(2-(3,4-epoxycyclohexyl)ethyl)methylsiloxane]; poly(vinyl chloride-co-isobutyl vinyl ether); poly(indene-co-coumarone); poly(styrene-co-4-bromostyrene-co-divinylbenzene); poly(ethylene-co-butyl acrylate-co-carbon monoxide); poly(vinyl acetate-co-butyl maleate-co-isobornyl acrylate) solution; poly(3,3′,4,4′-benzophenonetetracarboxylic dianhydride-co-4,4′-oxydianiline/1,3-phenylenediamine), amic acid (solution); poly(tetrafluoroethylene-co-vinylidene fluoride-co-propylene); poly(ethylene-co-methacrylic acid) lithium salt; poly(styrene-co-butadiene-co-methyl methacrylate); poly(vinylidene chloride-co-vinyl chloride); poly(styrene-co-maleic acid), partial isobutyl ester; poly[4,4′-methylenebis(phenyl isocyanate)-alt-1,4-butanediol/poly(ethylene glycol-co-propylene glycol/polycaprolactone]; poly(ethylene-co-methacrylic acid); poly(isobutylene-co-maleic acid) sodium salt; poly(ethylene-co-methacrylic acid) zinc salt; poly(4-styrenesulfonic acid-co-maleic acid) sodium salt; poly(acrylonitrile-co-butadiene-co-acrylic acid), glycidyl methacrylate diester; poly(urea-co-formaldehyde), butylated solution; poly(ethylene-co-methyl acrylate-co-glycidyl methacrylate); poly[(phenyl glycidyl ether)-co-dicyclopentadiene]; poly[(o-cresyl glycidyl ether)-co-formaldehyde]; poly(urea-co-formaldehyde), methylated; poly(acrylic acid-co-maleic acid) solution; poly(3-hydroxybutyric acid-co-3-hydroxyvaleric acid); poly(p-toluenesulfonamide-co-formaldehyde); poly(styrene-co-allyl alcohol); poly(2-acrylamido-2-methyl-1-propanesulfonic acid-co-styrene); poly(acrylonitrile-co-butadiene); poly(4-vinylphenol-co-methyl methacrylate); poly[dimethylsiloxane-co-methyl(3-hydroxypropyl)siloxane]-graft-poly(ethylene-ran-propylene glycol) methyl ether; poly(hexafluoropropylene oxide-co-difluoromethylene oxide)monoamidosilane; poly(dimethylamine-co-epichlorohydrin-co-ethylenediamine) solution; poly(ethylene-co-butyl acrylate-co-maleic anhydride); poly(trimellitic anhydride chloride-co-4,4′-methylenedianiline); poly[methylmethacrylate-co-(Disperse Orange 3 acrylamide)]; poly[((S)-( [symbol] )(4-Nitrophenyl)-2-pyrrolidinemethyl)methacrylate-co-methylmethacrylate]; poly[(propylmethacryl-heptaisobutyl-PSS)-co-(t-butylmethacrylate)]; poly[(methylmethacrylate)-co-(2-naphthyl methacrylate)]; poly[methylmethacrylate-co-(fluoresceinO-acrylate)]; poly[methylmethacrylate-co-(fluoresceinO-methacrylate)]; poly{[2-[2′,5′-bis(2″-ethylhexyloxy)phenyl]-1,4-phenylenevinylene]-co-[2-methoxy-5-(2′-ethylhexyloxy)-1,4-phenylenevinylene]}; poly[(methylmethacrylate)-co-(Disperse Red 1 acrylate)]; poly(4-hydroxy benzoic acid-co-ethylene terephthalate); poly(vinylidene chloride-co-acrylonitrile); poly(dimethylsiloxane-co-diphenylsiloxane), dihydroxy terminated; poly(1,4-butylene adipate-co-1,4-butylene succinate), extended with 1,6-diisocyanatohexane; poly(dicyclopentadiene-co-p-cresol); poly[ethyl acrylate-co-methacrylic acid-co-3-(1-isocyanato-1-methylethyl)-α-methylstyrene], adduct with ethoxylated nonylphenol solution; poly(epichlorohydrin-co-ethylene oxide); poly(Bisphenol A-co-4-nitrophthalic anhydride-co-1,3-phenylenediamine); poly(ethylene-co-methyl acrylate-co-acrylic acid); poly(propylene-co-1-butene); Nylon 6/66; poly(ethylene-co-acrylic acid) sodium salt; poly(ethylene-co-vinyl acetate-co-carbon monoxide); poly(melamine-co-formaldehyde), methylated/butylated (55/45); poly(maleic acid-co-olefin) sodium salt solution; poly(tetrafluoroethylene oxide-co-difluoromethylene oxide) α,ω-diisocyanate; poly(lauryl methacrylate-co-ethylene glycol dimethacrylate); poly[(phenyl isocyanate)-co-formaldehyde]; poly[2,6-bis(hydroxymethyl)-4-methylphenol-co-4-hydroxybenzoic acid]; poly(tetrafluoroethylene oxide-co-difluoromethylene oxide) α,ω-dicarboxylic acid; poly[methylmethacrylate-co-(Disperse yellow 7 acrylate)]; poly[(methylmethacrylate)-co-(9H-carbazole-9-ethylacrylate)]; poly[methylmethacrylate-co-(N-(1-naphthyl)-N-phenylmethacrylamide)]; poly[(methylmethacrylate)-co-(Disperse Red 1 methacrylate)]; poly(L-lactide-co-caprolactone-co-glycolide); poly[methylmethacrylate-co-(7-(4-trifluoromethyl)coumarin acrylamide)]; poly[dimethylsiloxane-co-methyl(3,3,3-trifluoropropyl)siloxane]; poly[dimethylsiloxane-co-methyl(stearoyloxyalkyl)siloxane]; poly(hexafluoropropylene oxide-co-difluoromethylene oxide) alcohol, ethoxylated phosphate; poly(ethylene-co-1,2-butylene)mono-ol; poly[dimethylsiloxane-co-methyl(3-hydroxypropyl)siloxane]-graft-tetrakis(1,2-butylene glycol); poly(1,4-butylene adipate-co-polycaprolactam); poly(vinyl acetate-co-crotonic acid); poly(tert-butyl acrylate-co-ethyl acrylate-co-methacrylic acid); poly(1-vinylpyrrolidone-co-styrene); poly(tetrafluoroethylene oxide-co-difluoromethylene oxide)-α,ω-bis(methyl carboxylate); poly(vinylidene chloride-co-methyl acrylate); poly(acrylonitrile-co-vinylidene chloride-co-methyl methacrylate); poly(styrene-co-maleic anhydride), partial cyclohexyl/isopropyl ester, cumene terminated; poly(4-ethylstyrene-co-divinylbenzene); poly(dimethylsiloxane-co-dimer acid), bis(perfluorododecyl) terminated; poly(styrene-co-maleic anhydride), partial propyl ester, cumene terminated; poly(dimer acid-co-ethylene glycol), hydrogenated; poly(ethylene-co-glycidyl methacrylate); poly[dimethylsiloxane-co-methyl(3-hydroxypropyl)siloxane]-graft-poly(ethylene glycol) 3-aminopropyl ether; poly(dimer acid-co-1,6-hexanediol-co-adipic acid), hydrogenated; poly(3,3′,4,4′-biphenyltetracarboxylic dianhydride-co-1,4-phenylenediamine), and amic acid solution; and poly[N,N′-bis(2,2,6,6-tetramethyl-4-piperidinyl)-1,6-hexanediamine-co-2,4-dichloro-6-morpholino-1,3,5-triazine].

In some embodiments, a single nanoparticle may be coated with block copolymers, or nanoparticle-polymer composites containing one or more nanoparticles may be usefully employed in the methods of the disclosure or as a probe or delivery vehicle.

Block copolymers can be used to control the degradation of nanoparticle. For example, block copolymers can be used to either protect (make bio-compatible) the nanoparticle against degradation in biological conditions, especially for in vivo applications, or control the degradation rate/degree of the nanostructure, by varying the molecular structure of the block copolymer.

The term “phospholipid” as used herein refers to, but is not limited to, phosphatidylcholine such as dilauroyl phosphatidylcholine, dimyristoylphosphatidylcholine, dipalmitoylphosphatidylcholine, distearoylphosphatidyl-choline, diarachidoylphosphatidylcholine, dioleoylphosphatidylcholine, dilinoleoyl-phosphatidylcholine, dierucoylphosphatidylcholine, palmitoyl-oleoyl-phosphatidylcholine, egg phosphatidylcholine, myristoyl-palmitoylphosphatidylcholine, palmitoyl-myristoyl-phosphatidylcholine, myristoyl-stearoylphosphatidylcholine, palmitoyl-stearoylphosphatidylcholine, stearoyl-palmitoylphosphatidylcholine, stearoyl-oleoyl-phosphatidylcholine, stearoyl-linoleoylphosphatidylcholine and palmitoyl-linoleoylphosphatidylcholine. Asymmetric phosphatidylcholines are referred to as 1-acyl, 2-acyl-sn-glycero-3-phosphocholines, wherein the acyl groups are different from each other. Symmetric phosphatidylcholines are referred to as 1,2-diacyl-sn-glycero-3-phosphocholines. The term “phosphatidylcholine” as used herein can refer to, such as, but is not limited to, phosphatidylcholine 1,2-dimyristoyl-sn-glycero-3-phosphocholine (DMPC); phosphatidylcholine 1,2-dioleoyl-sn-glycero-3-phosphocholine is abbreviated herein as “DOPC.”

In general, saturated acyl groups found in various lipids include groups having the trivial names propionyl, butanoyl, pentanoyl, caproyl, heptanoyl, capryloyl, nonanoyl, capryl, undecanoyl, lauroyl, tridecanoyl, myristoyl, pentadecanoyl, palmitoyl, phytanoyl, heptadecanoyl, stearoyl, nonadecanoyl, arachidoyl, heniecosanoyl, behenoyl, trucisanoyl and lignoceroyl. The corresponding IUPAC names for saturated acyl groups are trianoic, tetranoic, pentanoic, hexanoic, heptanoic, octanoic, nonanoic, decanoic, undecanoic, dodecanoic, tridecanoic, tetradecanoic, pentadecanoic, hexadecanoic, 3,7,11,15-tetramethylhexadecanoic, heptadecanoic, octadecanoic, nonadecanoic, eicosanoic, heneicosanoic, docosanoic, trocosanoic and tetracosanoic. Unsaturated acyl groups found in both symmetric and assymmetric phosphatidylcholines include myristoleoyl, palmitoleoyl, oleoyl, elaidoyl, linoleoyl, linolenoyl, eicosenoyl and arachidonoyl. The corresponding IUPAC names for unsaturated acyl groups are 9-cis-tetradecanoic, 9-cis-hexadecanoic, 9-cis-octadecanoic, 9-trans-octadecanoic, 9-cis-12-cis-octadecadienoic, 9-cis-12-cis-15-cis-octadecatrienoic, 11-cis-eicosenoic and 5-cis-8-cis-11-cis-14-cis-eicosatetraenoic.

Phosphatidylethanolamines include, but are not limited to, dimyristoyl-phosphatidylethanolamine, dipalmitoyl-phosphatidylethanolamine, distearoyl-phosphatidylethanolamine, dioleoyl-phosphatidylethanolamine and egg phosphatidylethanolamine. Phosphatidylethanolamines may also be referred to under IUPAC naming systems as 1,2-diacyl-sn-glycero-3-phosphoethanolamines or 1-acyl-2-acyl-sn-glycero-3-phosphoeihanolamine, depending on whether they are symmetric or asymmetric lipids.

Suitable sphingomyelins for inclusion in the nanoparticles of the present disclosure include brain sphingomyelin, egg sphingomyelin, dipalmitoyl sphingomyelin, and distearoyl sphingomyelin.

Suitable lipids for use in the present invention will have sufficient long-term stability to achieve an adequate shelf-life. Factors affecting lipid stability are well-known to those of skill in the art and include factors such as the source (e.g. synthetic or tissue-derived), degree of saturation and method of storage of the lipid. It is further contemplated that the phospholipid may be conjugated to a moiety such as a labeling moiety, a chelating agent for the attachment to the nanoparticle of a metallic labeling ion, and the like.

The term “celastrol” as used herein refers to a triterpenoid antioxidant compound isolated from the Chinese Thunder of God vine (T. wilfordii) having about 15 times the antioxidant potency of α-tocopherol. Celastrol has the formal chemical name of 3-hydroxy-9β,13α-dimethyl-2-oxo-24,25,26-trinoroleana-1(10),3,5,7-tetraen-29-oic acid.

### DESCRIPTION

The disclosure encompasses embodiments of a novel liposomal nanoparticle that has been engineered to be particularly useful for the delivery of compounds to cells found in the peripheral nervous system, endothelial cells that form the blood brain barrier, and epithelial cells that line the brain choroid plexus. Accordingly, the nanoparticles of the disclosure are intended to be useful for the delivery of compounds suitable for therapeutic purposes and/or imaging contrast agents that may not otherwise gain access to neuronal axons, or regions of the brain.

It has been found particularly advantageous for the purpose of targeting neuronal cells and endothelial cells of the blood vessels that serve the brain, to include cholesterol as a component of the liposomal nanoparticles and which that surprisingly increases the affinity of the nanoparticles for such as Schwann cells, glial cells, and the like. Furthermore, besides the delivery of therapeutic agents, it has been found that image contrast agents, such as those suitable for use in MRI techniques may also be delivered to neural cells, including of the peripheral nervous system, thereby providing a usefully specific means of targeting such cells.

The liposomal nanoparticles of the disclosure, therefore, comprise a phospholipid, a non-ionic surfactant, and cholesterol (or a derivative thereof). It is, however, contemplated that the nanoparticles of the disclosure may usefully incorporate such components as block co-polymers, or any phospholipid that maintains the relative specificity of the nanoparticles for such as Schwann and glial cells and/or the endothelial cells that form the blood-brain barrier and/or the epithelial cells that line the brain choroid plexus.

The efficient delivery of diagnostic and therapeutic compounds to specific cells of the nervous system is of great importance as neural disorders often initiate in certain cell populations. Nanoparticles engineered from specific lipids provide a unique opportunity to achieve this task as the composition of the particles can be engineered to facilitate entry into specific cell types. Liposome technology provides a promising approach to target cells of the nervous system and it has been the topic of many studies (Spuch & Navarro (2011) J. Drug Delivery). Yet the application of this technology against diseases of the central nervous system such as Alzheimer's and Parkinson's diseases pose unique challenges due to the barrier properties of brain endothelia which forms the blood-brain-barrier (BBB) (Spuch and Navarro, 2011). However, in studies where diseases of the CNS or other organs of the body were targeted, a number of positive properties of liposomes have been revealed, including low toxicity, high carrier capacity, sustained drug release, stability within the plasma and lack of immune system activation. In addition, liposomes can be made up of natural lipid components therefore pose low toxicity risk. Neurons and glial cells have specific membrane properties that can be utilized to facilitate the uptake of lipid nanoparticles, namely the endocytic pathway.

Myelinating glial cells of the peripheral nervous system (PNS), called Schwann cells, can give rise to degenerative neurological disorders, such as demyelinating neuropathies. These disorders initiate in the glial cells and lead to axonal and muscle atrophy. Due to the extensive lengths of the myelin sheath, delivery of small molecules to myelinating glial cells of the PNS poses a great therapeutic obstacle. In the absence of normal glial myelin, the neuronal signal does not reach the target organ, such as skeletal muscle, which leads to functional deficits in humans and mice. This demyelinating disease phenotype can be reproduced in explant cultures from neuropathic mice and used to test therapeutics that have the potential to correct the defect.

In addition, Schwann cells play critical roles in synaptic biology, including at the neuromuscular junction. Having the ability to identify and target myelinating and synaptic (non-myelinating) Schwann cells will provide a large number of opportunities for staging disease, as well as alleviating disease phenotypes. In an ex vivo diaphragm preparation, it was also found that the neurophilic nanoparticles of the disclosure could be targeted to motor neuron terminals, which allowed the assessment of the integrity of neuromuscular junction in disorders such as Pompe's disease and ALS. In addition, axonal neuropathies such as CMT type II also involve alterations of the neuromuscular synapses, therefore disease staging as well as the delivery of neuroprotective compounds to affected axons are applications for the neurophilic liposomes of the disclosure.

The neurophilic nanoparticles of the disclosure may be taken up by Schwann cells (which are glial cells of the peripheral nervous system), neuronal cells, and brain microvascular endothelial cells that comprise the blood brain barrier.

Liposomes are taken up by cells primarily via the endocytosis or transcytosis process. Endocytosis/transcytosis can occur through different mechanisms and it is an active process in neurons and Schwann cells. In neurons, synaptic vesicles are recycled by the endocytic pathway. Thus, endocytosis is an active process in all neurons that use chemical synapses, including sensory and motor neurons of the PNS. In neurons, it has been shown that endocytosis at the presynaptic terminal is mediated by the caveolae which are cholesterol-rich lipid rafts characterized by the presence of the protein caveolin. The role of endocytosis in Schwann cells is less studied, yet the expression of the caveolae protein caveolin-1 parallels post-natal peripheral nerve development and myelination. Recent studies in oligodendrocytes, as well as in Schwann cells, indicate that the endocytic pathway is involved in membrane remodeling during myelination. In agreement, a recent report shows the dependence of peripheral nerve remyelination on an intact lysosomal system, further supporting a role for the endocytic pathway in peripheral nerve biology. Accordingly, by adjusting the liposomes of the disclosure for uptake by the endocytic pathway, they can be targeted effectively to presynaptic nerve terminals and Schwann cells, including myelinating cells.

Liposomes were selected to constitute the nanoparticles of the disclosure. Liposomes are vesicles made of phospholipid bilayers as disclosed, for example, in Spuch & Navarro (2011) J. Drug Delivery. Liposomes can be filled with drugs, and have been used to deliver drugs to treat various diseases, including fungal infections, cancer, and Hepatitis A infections. Because liposomes are comprised of hydrophobic membranes surrounding spaces of aqueous solution, they can carry hydrophobic molecules (within the hydrophobic membranes) and hydrophilic molecules (within the aqueous solution spaces) to the diseased sites. Furthermore, liposomes can be manipulated into various sizes to facilitate the drug delivery process.

In living organisms, cells take up liposomes primarily via the endocytosis process, which is a way for cells to engulf macromolecules. Endocytosis can occur through different mechanisms. One such mechanism is mediated by the caveolae, which are non-clathrin-coated plasma membrane buds that exist on the surface of many cell types, including neurons, glial cells, and endothelial cells. Caveolae are special type of lipid rafts that are rich in proteins as well as cholesterol.

The present disclosure provides embodiments of a liposomal nanoparticle having affinity for neuronal cells, the endothelial cells that constitute the blood brain barrier and the epithelial cells that line the brain choroid plexus. It has been found that the combination of a phospholipid, a non-ionic surfactant, and cholesterol can form nanoparticles useful for the delivery of compounds such as, but not limited to, small molecules, therapeutic agents, labeling moieties, oligonucleotides, and the like, to neural cells, and especially to the neural cells of the peripheral neuronal system. The vehicles of the disclosure are also useful for the delivery of such agents to endothelial cells that constitute the blood-brain barrier and epithelial cells that line the brain choroid plexus, thereby being capable of delivering the agents to the neural tissue of the brain.

It is contemplated to be within the scope of the present disclosure for the phospholipid component of the nanoparticles to be any such species as described herein, or a combination of phospholipid species that alone or in combination can form bilayer liposomal nanoparticles. Thus, an especially useful, but not a limiting example, of a nanoparticle of the disclosure comprises the phospholipid DOPC, Poloxamer 188, and cholesterol. It is contemplated, however, that phospholipids other than DOPC, such as 1,2-dioleolyl-sn-glycero-3-phosphoethanolamine, may be incorporated into the nanoparticles either alone or in combination with, for example, DOPC. In some embodiments of the nanoparticles, at least one of the phospholipid components may have attached thereto a detectable label, e.g. a fluorescent label, a therapeutic agent or any other compound desired to be delivered to a neural cell. When introduced to a cell or an animal, the label allows tracing of the liposomal nanoparticles within the recipient cell, a tissue, a neuronal network, and the like, thereby permitting an assessment of the efficiency of delivery of the nanoparticles to a target, its stability in vivo or in vitro, or to provide a method of imaging a target cell or tissue to detect such as neuropathies, abnormal cell function, and the like.

Useful as non-ionic surfactants are block copolymers, of which Poloxamer 188 is an especially useful species for directing the nanoparticles of the disclosure to be taken up by a target cell, in particular by endocytosis by neuronal cells. It is, however, contemplated that a diverse variety of copolymers including the block copolymers as listed in the present disclosure may be usefully incorporated in the nanoparticles of the disclosure.

In the various embodiments of the nanoparticles of the disclosure, phospholipid dioleoyl-phosphatidylcholine (DOPC) can be used to incorporate the liposome nanoparticles, although it is intended that the phospholipid is not limited to DOPC. DOPC is biocompatible because it is a major constituent of cell membranes. DOPC has a very low “liquid-to-gel” transition temperature, making it highly versatile and easy to manipulate. To construct the neurophilic nanoparticles, DOPC may be mixed with different surfactants such as, but not limited to Tween-20, Tween-80, and Poloxamer 188, and the like. Surfactants, being non-ionic, are compatible with lipids and are mainly used as emulsifiers in foods, cosmetics, and pharmaceuticals. DOPC may be combined with Tween-80 or Poloxamer 188, and the like, so that the neurophilic nanoparticles can more readily cross the blood brain barrier and enter the central nervous system, or cross the blood nerve barrier and enter the peripheral nervous system. Poloxamers, also known as PLURONICS®, are non-ionic tri-block polymers composed of a block of central hydrophobic polypropylene oxide unit flanked by hydrophilic polyethylene oxide block units. Poloxamer 188 is composed of 30 polypropylene oxide units flanked by 75 polyethylene oxide units on each side.

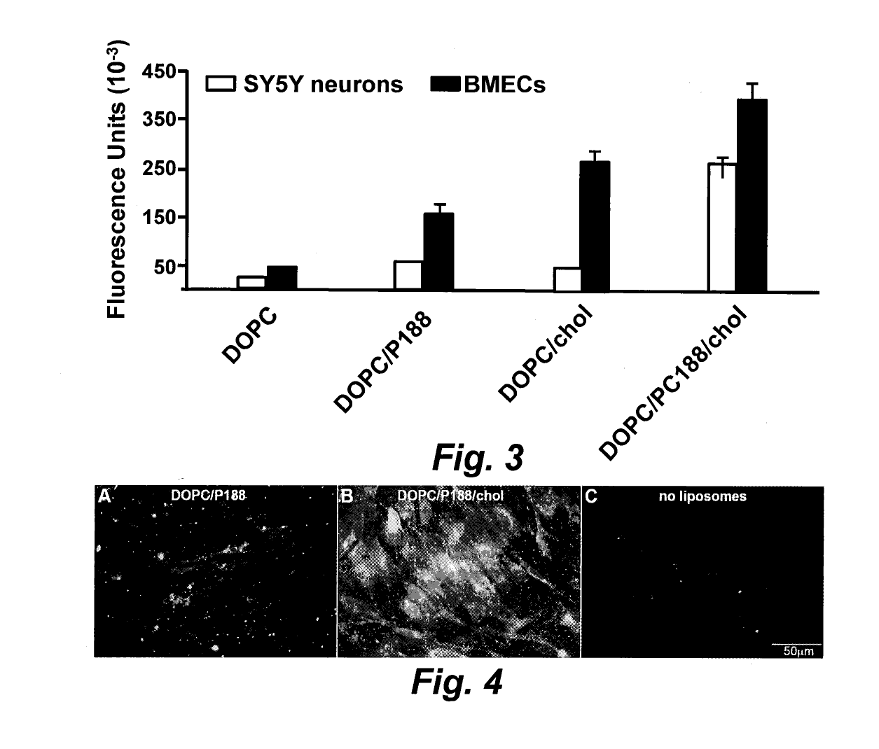

As shown in FIGS. 3A and 3B, P188 enhances liposome uptake by human SY5Y neurons and human brain microvascular endothelial cells (BMECs). To stimulate caveolae-mediated endocytosis/transcytosis, cholesterol was added to the liposome formulations to obtain the novel DOPC/P188/cholesterol liposomes of the disclosure. As shown in FIG. 3, highest liposome uptake was observed with DOPC/P188/cholesterol liposomes for SY5Y neurons and BMECs, about 11- and 8-fold higher than that of conventional DOPC liposomes. These studies indicate that the composition of liposomes greatly influences their uptake by cells.

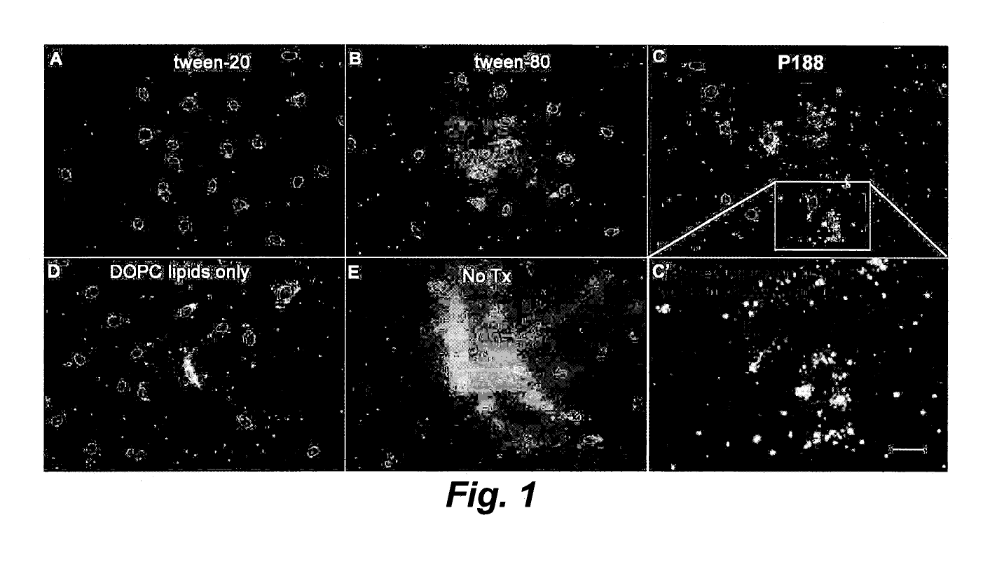

The uptake of basic DOPC liposome formulations by non-myelinating Schwann cells cultured from neonatal rat pups was also demonstrated. As shown in FIGS. 1A-1E, the addition of P188 enhanced the uptake of the DOPC liposomes after an 8-hour incubation, while Tween-20 or Tween-80 did not have an obvious effect (FIG. 1, Panels A and B). Similar results were obtained by FACS analysis of the cells. These initial studies also established that liposomes are not toxic to the Schwann cells as judged from the intact Hoechst nuclear dye labeling.

Cholesterol was added to the lipid formulation, and the incubation time with the liposomes was increased from 8 h to 24 h at 37° C. As shown in FIG. 4, the addition of cholesterol to the P-188 formulation robustly improved the uptake of liposomes by the Schwann cells, without apparent cellular toxicity. These studies in non-myelinating rat Schwann cells confirm that liposome formulations can be optimized for uptake into primary glial cells.

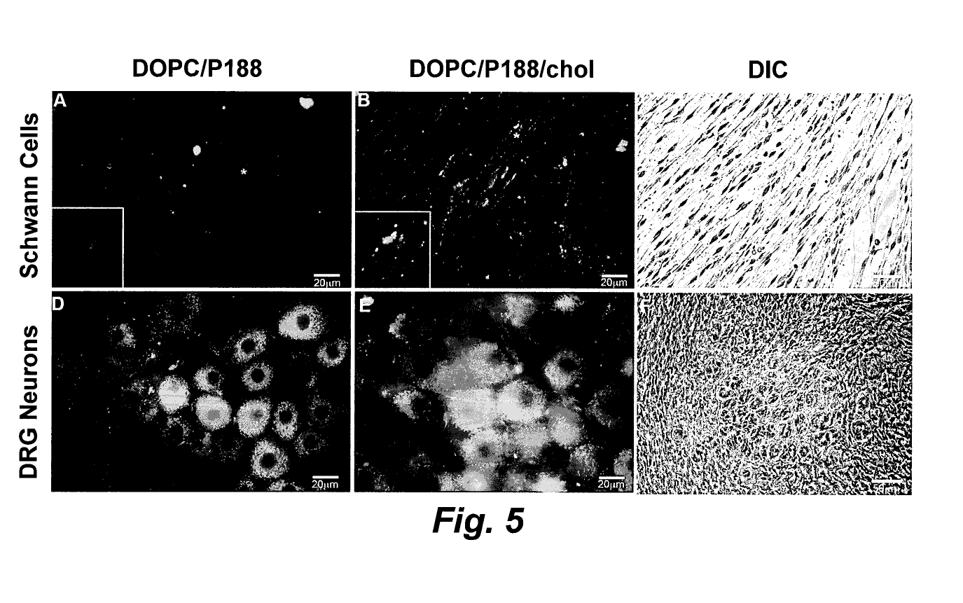

The uptake of DOPC/P188/cholesterol liposomes by dorsal root ganglion (DRG) explant cultures, which contain sensory neurons as well as Schwann cells, was also demonstrated, as shown in FIG. 5). Similar to the experiments in isolated non-myelinating Schwann cells (FIGS. 1 and 4) the uptake of liposomes by glial cells was notably enhanced by cholesterol (compare FIG. 5, Panels A and B). In contrast, both green fluorescent DOPC/P188 and DOPC/P188/cholesterol liposomes were avidly taken up by the sensory neurons and accumulate within the neuronal cell bodies (FIG. 5, Panels D and E). Panels C and F of FIG. 5 show lower magnification views of the cultures under phase contrast viewing.

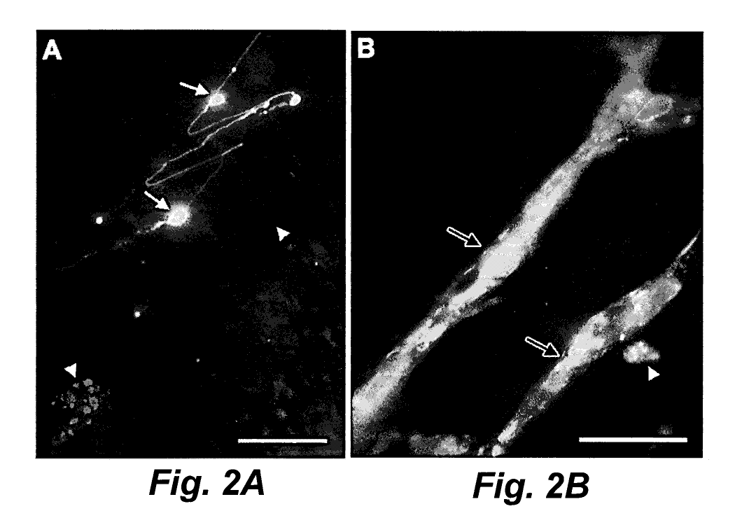

The uptake of DOPC/P188/cholesterol liposomes by neurons and myelinating Schwann cells was also studied using ex vivo diaphragm preparation. The response of acutely-isolated neural tissues to compounds or toxins can be evaluated ex vivo, an approach that has been utilized in studies of neurodegenerative disorders (Cho et al. 2007). Diaphragms from 1-month old mice were dissected into cold HBSS and transferred to DMEM supplemented with 10% FCS and 200 μM liposomes. To label post-synaptic motor end plates, α-bungaroxin (1 μM) was added for the last 30 minutes, followed by fixation. FIGS. 2A and 2B illustrate that green fluorescent DOPC/P188/Cholesterol liposomes were internalized within thin axonal processes (FIG. 2A, arrows) near post-synaptic motor end plates identified with a α-bungarotoxin; and by myelinating Schwann cells (FIG. 2B, arrowheads). These results indicate that DOPC/P188/cholesterol liposomes provide viable options for optimization for uptake into neurons, including motor neurons innervating the diaphragm and make them suitable for in vivo application.

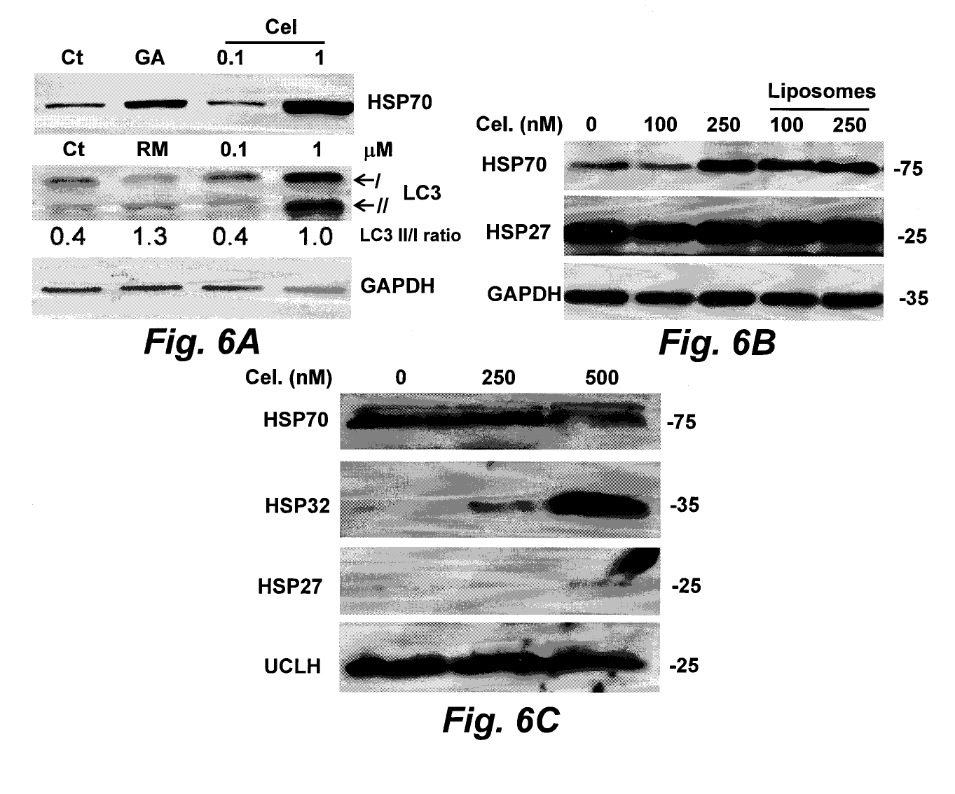

Treatment of Dorsal Root Ganglion Explant Cultures from Mice by Drug-Carrying Liposomes:
The main goal of therapeutic approaches for neuropathies is to improve myelination of the nerves and neuromuscular function. Elevation of chaperones, and/or autophagy can be expected to enhance myelin synthesis by neuropathic samples, as compared to untreated controls (Rangaraju et al., (2008) Neurobiol. Dis. 32: 105-115; Rangaraju et al., (2010) J. Neurosci. 30: 11388-11397). Small hydrophobic molecules, such as celastrol, resveratrol, rapamycin, known to enhance protein chaperone or protein degradation pathways have been identified as possible therapeutic candidates for demyelinating neuropathies (Zhang & Sarge (2007) J. Mol. Med. 85: 1421-1428; Rangaraju et al., (2008) Neurobiol. Dis. 32: 105-115; Rangaraju et al., (2010) J. Neurosci. 30: 11388-11397). FIG. 6A shows that celastrol dose-dependently induced heat shock protein 70 (HSP70) expression and increased the ratio of the authophagy proteins LC3 II to LCI in rat Schwann cells. FIG. 6B shows that free celastrol, at 250 nM, but not 100 nM, induced mouse Schwann cells co-incubated with mouse dorsal root ganglion to increase HSP70 expression. However, celastrol-filled liposomes induced mouse Schwann cells to increase HSP70 expression at both 100 and 250 nM concentrations (FIG. 6B). Free celastrol or celastrol-filled liposomes did not affect HSP27 expression. Increased HSP70 levels were produced by Schwann cells, since HSP70 expression was not affected by the celastrol treatments when dorsal root ganglion were depleted of Schwann cells, as shown in FIG. 6C. These data indicate that the neurophilic liposomes can enhance celastrol's ability in selectively inducing the chaperone pathway.

The liposomal nanoparticles of the disclosure are also advantageous for use as delivery vehicles for the delivery of imaging contrast agents to neuronal cells or neural tissues of an animal or human subject. For example, but not intended to be limiting, the nanoparticles of the disclosure may have attached thereto, or encapsulate, a detectable moiety desired to be delivered to the subject. Suitable detectable moieties include such as fluorescent labels, metallic moieties and the like that are useful in one or more imaging procedures. Thus the liposomal nanoparticles of the disclosure may include gadolinium or a modified form thereof that is suitable as an imaging contrast agent such as in MRI techniques. Such detectable labels, when combined with a delivery vehicle as disclosed herein allow those of skill in the art to obtain images of the neural cells and, if the nanoparticles further include a therapeutic agent(s) to track the progress of the said agent within a recipient animal or human subject, including localization within the subject's body and removal of the agent subsequently.

One aspect of the present disclosure, therefore, encompasses embodiments of a liposomal nanoparticle vehicle for the delivery of a compound to a neuronal cell or a glial cell, where said vehicle comprises: a phospholipid; a non-ionic surfactant; and cholesterol.

In the embodiments of this aspect of the disclosure, the phospholipid can be selected from dioleoyl-phosphatidylcholine (DOPC), 1,2-dioleolyl-sn-glycero-3-phosphoethanolamine, or a combination thereof.

In the embodiments of this aspect of the disclosure, the non-ionic surfactant can be a block copolymer.

In the embodiments of this aspect of the disclosure, the block copolymer can be a tri-block copolymer and can have the formula (PEO)₇₅—(PPO)₃₀—(PEO)₇₅.

In the embodiments of this aspect of the disclosure, the vehicle can further comprise a compound desired to be delivered to a neuronal cell or a glial cell and/or to cross the blood-brain barrier.

In some embodiments of this aspect of the disclosure, the compound can be a detectable label or a therapeutic agent.

In embodiments of this aspect of the disclosure, the detectable label is a fluorescent label, a detectable metal-based label, a radiolabel, or an imaging agent.

In embodiments of this aspect of the disclosure, the imaging agent is gadolinium, or a derivatized variant thereof.

In embodiments of this aspect of the disclosure, the imaging agent is Gadolinium-F (Gf) or Gadolinium-M.

In embodiments of this aspect of the disclosure, the imaging agent is Gadolinium-F (Gf).

In the embodiments of this aspect of the disclosure, the nanoparticle can have a diameter between about 50 nm to about 700 nm.

In some embodiments of this aspect of the disclosure, the gadolinium is bound to a chelating agent and said chelating agent is conjugated to a lipid.

Another aspect of the disclosure encompasses embodiments of a method of imaging a neuronal cell or a glial cell or a system of neuronal cells, the method comprising delivering to a recipient animal or human subject a liposomal nanoparticle vehicle according to the disclosure, allowing the vehicle to deliver the detectable label or imaging agent to a neuronal cell or a glial cell, and detect the label in the recipient neuronal cell or system of neuronal cells or a glial cells.

Another aspect of the disclosure encompasses embodiments of a method of diagnosing a neuropathological condition in an animal or human subject, the method comprising delivering to the animal or human subject a liposomal nanoparticle vehicle comprising a detectable label, allowing the vehicle to access peripheral neurons or peripheral glial cells of the recipient subject, detecting the label in the peripheral neural system of the subject, thereby determining the presence or absence of a neuromuscular pathology of the subject animal or human.

Still another aspect of the disclosure encompasses embodiments of a method of delivering a therapeutic agent to a neuronal cell or glial cell or a system of neuronal cells and glial cells, the method comprising contacting a recipient cell or a system of neuronal cells with an effective amount of a liposomal nanoparticle vehicle according to the disclosure, wherein the liposomal nanoparticle vehicle comprises a therapeutic agent characterized as modulating a bioactivity of the recipient cell or tissue, and allowing the vehicle to deliver the therapeutic agent to the neuronal cell or glial cell or a system of neuronal cells and glial cells, whereby the therapeutic agent modulates a bioactivity of the recipient cell or tissue.

In the embodiments of this aspect of the disclosure, the liposomal nanoparticle vehicle can be formulated in a pharmaceutically acceptable composition.

The specific examples below are to be construed as merely illustrative, and not limitative of the remainder of the disclosure in any way whatsoever. Without further elaboration, it is believed that one skilled in the art can, based on the description herein, utilize the present disclosure to its fullest extent. All publications recited herein are hereby incorporated by reference in their entirety.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure, and the present disclosure and protected by the following claims.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### EXAMPLES

### Example 1

### Reagents

DOPC, cholesterol, 1,2-dioleoyl-sn-glycero-3-phosphoethanolamine-N-(7-nitro-2-1,3-benzoxadiazol-4-yl), and 1,2-dioleoyl-sn-glycero-3-phosphoethanolamine-N-(carboxyfluorescein) were purchased from Avanti Polar Lipids, Inc. Surfactants, such as Tween-20, Tween-80 and Poloxamer 188, were purchased from Sigma-Aldrich. Tertiary butanol was purchased from Fisher Scientific Co.

### Example 2

### Preparation of Liposomes

Liposome nanoparticles were prepared by a one-step process. DOPC was mixed with surfactants at different ratios ranging 0 to 50 percent. Excess tertiary butanol (3 mL) was added to the lipid/surfactant mixtures, frozen at −80° C. overnight, and lyophilized. The lyophilizates were stored at −20° C. until ready for use. Typically, the lyophilizates were used within 1 month of storage. When ready to use, the lipid/surfactant lyophilizates were warmed at room temperature for 10 to 15 min. Phosphate buffered saline, or cell culture medium, was used to reconstitute the lyophilizates into liposomes.

For analysis by FACS and microscopy, fluorescent liposomes were prepared as described above, except that fluorescent phospholipids, such as 1,2-dioleoyl-sn-glycero-3-phosphoethanolamine-N-carboxyfluorescein (CF-PE), could be added to the lipid mixture at a 2 mole percent final concentration before freezing and lyophilizing. This method allowed following the uptake and retention of liposomes within the target cells.

### Example 3

### Cellular Uptake of Liposomes

(a) Fluorescent Microscopy:

As established primary neuron cultures, such as the DRG neurons, are not suitable for FACS analysis, the uptake of liposome into these cells was evaluated by fluorescence microscopy. DRG and Schwann cells were incubated with fluorescent liposomes in DMEM containing 2% FBS for up to 24 hours.

Diaphragm was excised from an euthanized 2-month old mouse and incubated with 200 μM liposomes overnight in DMEM containing 10% FBS at 37° C. Post-synaptic motor end plates were visualized by addition of α-bungarotoxin-594 (2 μM) during the last hour of liposome incubation. Tissue was fixed with 4% paraformaldehyde in PBS for 10 minutes, washed 3 times with PBS and incubated with 1/2000 Hoescht dye, mounted and viewed under 60× and 100× oil immersion lenses with a Nikon Eclipse E800 fluorescent microscope. Using this approach the uptake of liposomes into presynaptic nerve terminals and myelinated Schwann cells was visualized, as shown in FIGS. 2A and 2B.

To study liposome subcellular localization, cell-permeant, red fluorescent organelle markers, including LYSOTRACKER®, ER-TRACKER®, and MITOTRACKER® (Invitrogen), were added to the cultures at the end of their incubation with fluorescein-labeled liposomes. LYSOTRACKER®, ER-TRACKER®, and MITOTRACKER® are used to label organelles, such as lysosomes, endoplasmic reticulum, and mitochondria, respectively, in live cells. Fluorescent anti-caveolin antibodies could also be used to co-localize liposomes with caveolae. In the myelinating explants cultures, myelinated Schwann cells could be identified with Fluoro-myelin stain or anti-myelin protein antibodies.

(b) Flow Cytometry:

Fluorescent liposomes were prepared as described above, except that fluorescent phospholipids such as 1,2-dioleoyl-sn-glycero-3-phosphoethanolamine-N-(7-nitro-2-1,3-benzoxadiazol-4-yl) (NBD-PE) or 1,2-dioleoyl-sn-glycero-3-phosphoethanolamine-N-(carboxyfluorescein) (CF-PE) were added to the lipid/surfactant mixture at a 2 mole percent final concentration before freezing and lyophilization. Cells were plated in 12-well plate in 0.8 to 1.0 ml medium. After overnight attachment, fluorescent liposomes were added to cells. Cells were incubated with fluorescent liposomes for up to 24 h at 37° C. Cells were trypsinized and washed. Cellular uptake of liposomes was measured by an Accuri C6 flow cytometer.

### Example 4

### Measurement of Cell Viability

While liposomes are known to have low toxicity their potential toxicity on neurons and Schwann cells could be evaluated using standard procedures. The Promega Celltiter 96 Aqueous nonradioactive proliferation (MTS) assay could be used to determine if the liposomes were toxic to these cells. The MTS assay is a colorimetric method for determining the number of viable cells in cytotoxicity assays. It is composed of the tetrazolium compound 3-(4,5-dimethylthiazol-2-yl)-5-(3-carboxymethoxyphenyl)-2-(4-sulfophenyl)-2H-tetrazolium, inner salt (MTS) and the electron coupling reagent phenazine methosulfate. MTS is reduced by cells into a formazan product that is soluble in tissue culture medium. The quantity of formazan product, measured by absorbance, is directly proportional to the number of living cells in culture. Cell viability can be expressed as (absorbance of treated cells/absorbance of untreated cells)×100%. Liposome formulations that induce pronounced toxicity (over 10% cell death) can be retested at lower concentrations for uptake and/or excluded from further experiments. Using fluorescence microscopy, the nuclear morphology of the liposome treated cells by the Hoechst dye can be monitored.

Cells were plated in 96-well plate in 0.10 to 0.15 ml medium. After overnight attachment, liposomes were added to cells in quadruplicate wells. Cells were incubated with liposomes for 5 to 6 days. At the end of the incubation, the CellTiter 96 Aqueous assay (Promega) was used to determine the effects of liposomes on cell viability. Cell viability was expressed as (absorbance of treated cells/absorbance of untreated cells)×100%.

### Example 5

### Schwann Cells Prefer Poloxamer 188-Containing Liposomes

Schwann cells are the principal glial cells of the peripheral nervous system and facilitate the conduction of action potentials along axons, nerve development and regeneration, synapse formation and function, and trophic support for neurons.

Primary Schwann cell cultures were established from newborn rat pups according to Fortun et al. (2007) Neurobiol Dis. 25: 252-265; Rangaraju et al., (2008) Neurobiol Dis. 32: 105-115, incorporated herein by reference in their entireties. Briefly, sciatic nerves can be dissociated enzymatically and mechanically and plated in normal growth medium (10% FCS/DMEM, supplemented with 20 μg/ml bovine pituitary extract (BTI) and 5 μM forskolin). From one neonatal rat litter enough cells were obtained for up to six experiments, each providing multiple 6-cm and two 24-well plates. Since the yields from rat samples are much higher as compared to mouse, initial experiments were performed in rat cells. Based on preliminary studies, DOPC/P188 liposomes compositions that are taken up by rat cells (FIG. 1, Panel C) are similarly effective in cultures from mice (FIG. 5, Panel A), and DOPC/P188/Cholesterol liposome compositions that are taken up by rat cells (FIG. 3, Panel B) are similarly effective in cultures from mice (FIG. 5, Panel B). The purity of the Schwann cell cultures was routinely evaluated by immunolabelling and were over about 90% as judged from anti-p75 and anti-S100β reactivity.

Dissociated dorsal root ganglion (DRG) neuron cultures were established from embryonic day 15 (E15) rodents (Amici et al., (2007) J. Neurosci Res. 85: 238-249; Rangaraju et al., (2008) Neurobiol Dis. 32: 105-115). These non-myelinating, dissociated cultures can serve to assess the uptake of liposomes into sensory neurons. 16 4-well dishes and over 18 6-cm dishes of neuronal cultures from about 20 rat embryos were established.

The uptake and subcellular fate of liposomes in myelinating DRG neuron explants cultures from rodent embryos could be evaluated. For these experiments, the Schwann cells were induced to proliferate and myelinate the DRG neuron networks, which typically occurs after 2-3 weeks of culture time. Since these cultures were grown on glass coverslips, they could be readily evaluated for the uptake and localization of the fluorescent liposomes.

Schwann cells were grown in DME medium containing 10% FBS, 5 μM forskolin, and 10 μg/ml bovine pituitary extract. Schwann cells were incubated with different DOPC liposome formulations carrying: Tween-20, 50% weight (FIG. 1, Panel A); Tween-80, 50% weight (FIG. 1, Panel B); Poloxamer 188, 50% weight (FIG. 1, Panel C); no surfactants (FIG. 1, Panel D). Fluorescent microscopy was used to study liposome uptake by Schwann cells. The nuclei of the Schwann cells were labeled with blue color Hoechst dye, while liposomes were labeled with the green carboxyfluorescein dye. The results in FIG. 1 show that Schwann cells internalized DOPC liposomes containing Poloxamer 188 (FIG. 1, Panel C). Schwann cells also internalized DOPC liposomes, which did not carry any surfactant, though not to the same extent as DOPC liposomes containing Poloxamer 188. DOPC liposomes containing Tween-20 or Tween-80 were not taken up by Schwann cells. These results indicated that DOPC liposomes containing Poloxamer 188 are preferentially taken up by Schwann cells.

Flow cytometry was then used to confirm the above observations. Schwann cells were incubated with liposomes at 150 and 300 μM concentrations. Percentages of Schwann cells taking up fluorescent DOPC liposomes ranged between 9 and 25%, while percentages of Schwann cells taking up fluorescent DOPC liposomes containing Poloxamer 188 ranged between 56 and 79%. Also, the median fluorescent intensity of Schwann cells was increased by about 2-fold when Poloxamer 188 was included in the DOPC formulation. These results confirm that Schwann cells prefer DOPC liposomes containing Poloxamer 188.

### Example 6

### SY5Y Brain Neuronal Cells Prefer Poloxamer 188-Containing Liposomes

Human SY5Y neuroblastoma cells, used as a model of human brain neurons, were cultured in DMEM/F12 medium containing 10% FBS. SY5Y cells were incubated with 200 μM of DOPC liposomes containing Tween-20 or DOPC liposomes containing Poloxamer 188. Flow cytometry showed that the percentages of SY5Y cells taking up DOPC liposomes containing 10% Tween-20 or DOPC liposomes containing 50% Tween-20 were 19% and 15%, respectively. The percentages of SY5Y cells taking up DOPC liposomes containing 10% Poloxamer 188 or DOPC liposomes containing 50% Poloxamer 188 were 16% and 51%, respectively. Also, the median fluorescent intensity of SY5Y cells was increased by about 10-fold when Poloxamer 188, instead of Tween-20, was added to the DOPC formulation at 50% weight ratio.

### Example 7

### Human Brain Microvascular Endothelial Cells (HBMEC) Take Up Poloxamer 188-Containing Liposomes

Immortalized human brain microvascular endothelial cells (HBMEC) that constitute the blood brain barrier were isolated from a brain biopsy of an adult female with epilepsy. HBMEC were subsequently immortalized by transfection with simian virus 40 large T antigen and maintained their morphologic and functional characteristics. Immortalized HBMEC were cultured in Medium 199 and supplemented with 10% FBS and 10% Nuserum.

HBMEC was incubated with 300 μM concentration of DOPC liposomes containing increasing weight of Poloxamer 188. The median fluorescent intensities of HBMEC incubated with 3000 μM liposomes containing 10, 30 or 50% poloxamer were 1.3-, 2.1-, and 3.6-fold of that of untreated cells. The percentages of HBMEC taking up liposomes containing 10%, 30% or 50% Poloxamer 188 were 50%, 55% and 58%, respectively.

### Example 8

### Poloxamer 188-Containing Liposomes are Not Toxic to SY5Y Cells or HBMEC

SY5Y cells and HBMEC were incubated with 0 to 400 μM liposomes for 5 to 6 days. There was no difference in cellular viabilities between untreated SY5Y cells and SY5Y cells incubated with DOPC liposomes containing 0, 10, 30, or 50% Poloxamer 188. Similarly, there was no difference in cellular viabilities between untreated HBMEC and HBMEC incubated with DOPC liposomes containing 0, 10, 30, or 50% Poloxamer 188. Therefore, DOPC liposomes containing up to 50% Poloxamer 188 were not toxic to SY5Y cells or HBMEC.

### Example 9

### Cholesterol Enhances HBMEC Uptake of Poloxamer 188-Containing Liposomes

DOPC liposomes were prepared containing 50% Poloxamer 188, and added 0%, 15% or 30% cholesterol to the liposomes. Flow cytometry results indicate that when HBMEC were incubated with 200 μM liposomes containing 0% cholesterol, the median fluorescent intensity of these cells was about 10-fold higher than that of untreated cells. When HBMEC were incubated with liposomes containing 15% and 30% cholesterol, their median fluorescent intensities were increased to 16-fold and 25-fold higher than that of untreated cells, respectively. Percentages of HBMEC taking up liposomes containing 0% cholesterol, 15% cholesterol, or 30% cholesterol were 40%, 51%, and 62%, respectively.

### Example 10

### Cholesterol Enhances Uptake of Poloxamer 188-Containing Liposomes by Schwann Cells, SY5Y Cells and HBMEC

The median fluorescent intensity of Schwann cells incubated with 300 μM liposomes containing 0% cholesterol was 7-fold higher than that of untreated cells. The median fluorescent intensities of Schwann cells incubated with 300 μM liposomes containing 15% cholesterol or 30% cholesterol were 12-fold and 24-fold higher than that of untreated cells, respectively.

Both Poloxamer 188 and cholesterol are required for avid nanoparticle uptake. The following DOPC liposome formulations were prepared: (a) formulation A: 0% Poloxamer 188, 0% cholesterol; (b) formulation B: 50% Poloxamer 188, 0% cholesterol; (c) formulation C: 0% Poloxamer 188, 30% cholesterol; and (d) formulation D: 50% Poloxamer 188, 30% cholesterol.

SY5Y cells were incubated with 150 μM liposomes. The median fluorescent intensity of SY5Y cells incubated with formulation A was 4-fold higher than that of untreated cells. The median fluorescent intensity of SY5Y cells incubated with formulation B was 6-fold higher than that of untreated cells. The median fluorescent intensity of SY5Y cells incubated with formulation C was also 6-fold higher than that of untreated cells. But the median fluorescent intensity of SY5Y cells incubated with formulation D was 9-fold higher than that of untreated cells. Percentages of SY5Y cells taking up formulations A, B, C, and D were 26%, 42%, 40%, and 53%, respectively.

Similar results were obtained with Schwann cells and HBMEC. Schwann cells were incubated with 300 μM liposomes. Percentages of Schwann cells taking up formulations A, B, C, and D were 25, 76, 41 and 94, respectively. HBMEC were incubated with 100 μM liposomes. The median fluorescent intensities of HBMEC incubated with formulations A, B, C, and D were 2.1-, 5.9-, 3.4- and 9.8-fold higher than that of untreated cells, respectively. Together, these results indicate that nanoparticle internalization by neuronal cells, Schwann cells and HBMEC was much higher when both Poloxamer 188 and cholesterol were included in the DOPC nanoparticle formulation.

### Example 11

Lipid-Mediated Delivery of Small Molecules to Myelinating Glial Cells of the Peripheral Nervous System can Correct the Neuropathic Phenotype in In Vitro Culture Models

The lyophilization method can be used to incorporate different types of drug molecules, including antisense oligonucleotides, into the liposomes can be used. Optimal liposomal drug formulation is defined as drug incorporation at least 70% and drug release in target cells, as detected by specific pathway biomarker.

Effective liposome-drug formulations can be identified by applying a specific biomarker for each drug and by increase in the number of myelin internodes on immunofluorescence microscopy and myelin protein expression by Western blots.

Lipid formulations according to the disclosure can enter into myelinating Schwann cells (FIGS. 1C, 2B, 4A, 4B, 5A, and 5B). Liposomes can be loaded with small hydrophobic compounds that are directed to enhance the protein chaperone, as shown in FIG. 6B, or the protein degradation pathways and have known biomarkers of activity.

These two pathways are targets for therapeutic intervention in demyelinating neuropathies. Compounds that could be usefully delivered with the selected liposome formulation can include such as, but is not limited to, curcumin, a naturally occurring hydrophobic molecule that has been shown to abrogate endoplasmic reticulum retention and aggregation of myelin proteins and celastrol, a naturally occurring triterpene which is known to activate chaperones and reduce protein mis-folding can be another compound, and autophagy activators. Perhexyline, niclosamide, carbamazepine, and resveratrol are all hydrophobic and are advantageous examples, but not considered limiting, of compounds for inclusion in the liposome formulations of the disclosure. Compounds delivered by liposomes can lead to in a more sustained pathway activation profile and will show lower toxicity even at high doses, as compared to directly applied “naked compound.”

Liposomes can be prepared in accordance with the methods of the present disclosure. Lipids can be added to drugs at 1-, 2-, 5-, 10- or 20-fold molar excess, to optimize liposomal drug formulations. The lipid/drug mixtures can be frozen and lyophilized. After reconstitution, free drugs can be separated from liposomal drugs by loading onto filter spin columns and centrifuged. HPLC-MS (UF Biomedical Mass Spectrophometry Core Facility) can be used to quantify the percentage of drug incorporation. This is done by measuring the amount of free drug in the filtrate/amount of total drug used in preparing the liposomes ×100%. Effective liposome formulations can be readily identified using known biomarkers for compound activity and improvement in myelination by the neuropathic samples.

### Example 12

### Myelinating DRG Explant Cultures from Neuropathic Mice

Three mouse models of PMP22 expression phenotypes will be used for these studies, including PMP22 Wt (+/+), PMP22 over-expressor C22 line (oe/+), and PMP22 mutant TrJ (TrJ/+) (Jackson laboratories). Breeder pairs of Wt males and affected females are housed under specific-pathogen-free conditions. This mating paradigm generates 50% Wt and 50% affected heterozygous embryos. All embryos used in these studies can be genotyped from genomic DNA. The TrJ mutation is detected using PCR and PMP22oe mice are identified by Southern blotting, or PCR. Mouse DRG explant cultures can be established from embryonic day (E) 13 pups similar to the techniques used for the rat. DRGs are isolated and treated with 0.25% trypsin (15 min, 37° C.). After centrifugation and several washes, small clumps of cells are plated on collagen coated glass coverslips in 10% FCS/high glucose MEM, supplemented with 100 ng/ml nerve growth factor. After 7 days in vitro (DIV) SC proliferation, elongation and ensheathment, myelin formation is induced with the addition of ascorbic acid (50 μg/ml). After an additional 1-3 weeks under myelinating conditions, cultures from Wt mice contain abundant myelin while neuropathic samples do not, allowing for easy detection of any improvement (Rangaraju et al., 2008 and 2010).

### Example 13

### Assessment of Drug Bioactivity and Myelin Synthesis after Liposome-Mediated Delivery

Drugs that are effectively internalized with the liposomes are likely to be released within the cytosol within 24 h (Spuch and Navarro, 2011). Drug release kinetics can be studied by using known biomarkers for each compound. For example, celastrol when directly applied to Schwann cells leads to a robust increase in Hsp70, as shown in FIGS. 6A and 6B.

The induction kinetics of directly applied compounds can be compared to the pattern obtained after lipid-mediated delivery. As shown in FIG. 6B, the direct application of 250 nM celastrol, but not 100 nM celastrol, to cultured Schwann cells led to robust activation of Hsp70 expression However, celastrol-filled liposomes induced mouse Schwann cells to increase HSP70 expression at both 100 and 250 nM concentrations (FIG. 6B). Elevation of chaperones can aid the trafficking of PMP22 (Wt copy) and interacting molecules to the cell membrane, and enhance myelin synthesis by neuropathic samples, as compared to untreated controls.

Enhancement of autophagy is expected to have a similar effect, with a potential increase in myelin internode length and a reduction in poly-ubiquitinated substrates, as was see with rapamycin. Myelin production in DRG neuron explant cultures can be measured 2 weeks after the initiation of myelination and liposome application, by biochemical assessments of myelin proteins, including protein zero (P0) and myelin basic protein (MBP). As Schwann cells do not synthesize significant amounts of MBP in the absence of myelin, the levels of MBP can be used as an indication of myelination. The bands corresponding to each myelin protein can be quantified using Scion Image software and graphed as percentage of protein made by empty liposome treated or Wt cultures, and the cultures by immunostaining with an anti-MBP antibody to label internodal myelin segments. The abundance of myelin internodes can be counted as the number of MBP+ internodal segments per 0.1 mm² area from three independent experiments, and eight random visual fields per condition. Internode lengths from the same experiments can be measured with Spot RT software (Diagnostic Instruments, Inc.). Measurements can be collected from three coverslips per genotype per liposome formulation. Statistical significance can be determined by Student's t-test using GraphPad Prism software. RM-treatment lengthens myelin internodes in normal Schwann cells, as well as cells from neuropathic mice.

### Example 14

### MR Imaging of Peripheral Nerves and Presynaptic Terminals

Clinical investigation of peripheral nerve lesions routinely involves nerve conduction studies and electromyography. Although these techniques allow the diagnosis of nerve damage and nerve conduction blocks, they cannot identify the underlying causes and location of nerve damage. Imaging studies such as MRI could be used to supplement nerve conduction studies and electromyography. MRI is a non-invasive technology that provides good contrast for soft tissues. Diethylenetriamine-pentaacetic acid (DTPA) chelated Gadolinium (Gd), an MRI contrast agent, allows changes in positive contrast readily observable. However, this conventional MRI technique is not sufficiently sensitive in identifying early signs of neurodegeneration, or early signs of nerve regeneration from drug treatments (Bar-Or et al. 2011; Zivadinov R 2007; Koltzenburg M and Bendszus M 2004). Also, much higher MRI resolution acquisition is needed in small animal models because neurodegeneration induced in small animals are typically of much smaller magnitudes than that in humans (Pirko and Johnson 2008). Thus, there is an urgent need to develop higher MRI resolution acquisition for preclinical and clinical studies of peripheral nerve lesions. We propose using neurophilic vehicles to deliver the Gd contrast agent to denervating muscles and peripheral nerves. With a high delivery payload, we should be able to increase T1-weighted MRI resolution sensitivity, and differentiate early signs of nerve degeneration and regeneration.

### Example 15

### Gadolinium-Filled Liposome Preparations

MRI, a non-invasive technology that provides good contrast can be used to visualize and quantify the disposition of the neurophilic liposomes in vivo. DTPA-Gd, an MRI contrast agent, allows changes in positive contrast readily observable. Lipid-conjugated DTPA-Gd can be purchased from Avanti Polar Lipids and incorporated into liposomes, at 10 mole percent, to form gadolinium-filled neurophilic liposomes. The Agilent 4.7T/200 MHz MRI spectrometer (UF Advanced Magnetic Resonance and Imaging Facility, AMRIS) can be used to characterize the longitudinal and transverse relaxation MRI time constants, T1 and T2, of the gadolinium-filled neurophilic liposomes incubated in PBS and serum. Gd-filled neurophilic liposomes, at Gd doses of 5, 10, 20 and 50 μmole per kg, can be injected into mouse diaphragm (four mice per dose). Mice can be imaged with the 4.7T/200 MHz MRI spectrometer at 15 min, 30 min, 1 h, 2 h and 4 h post-liposome administration. T1-weighted MR images of Schwann cells in the diaphragm can be obtained.

To obtain T1-weighted MR images of the peripheral nerves, mice (four per dose) can be injected intravenously (iv) with Gd-filled neurophilic liposomes, at Gd doses of 5, 10, 20 and 50 μmole per kg, and imaged with the 4.7T/200 MHz MRI spectrometer at 15 min, 30 min, 1 h, 2 h and 4 h post-liposome administration (Straathof et al. 2011).

### Example 16

### Liposome Uptake by Primary Cortical Neural Cultures

Neurophilic liposomes internalized by dorsal root ganglion neurons, which are located in the spine, has been demonstrated. To determine whether the neurophilic liposomes are taken up by cortical (brain) neurons, mixed cortical neurons harvested from postnatal day 0-1 mouse pups (Echeverria et al., (2005) Ann. N.Y. Acad. Sci. 1053: 460-471) were used. Mixed cortical neurons were plated and maintained in Neurobasal media containing B27 and 2% fetal bovine serum, 60 μM L-glutamine, 60 μM Glutamax, and antibacterial agents. At 4 DIV, cells were incubated with carboxyfluorescein-labeled DOPC/P188/Cholesterol liposomes for 24 h at 37° C. Lysotracker Red was added during the last 30-min incubation. Coverslips were rinsed in PBS, fixed with 4% paraformaldehyde, and nuclei were stained using Hoescht dye (in blue). Pictures were taken using an Olympus IX81-DSU Spinning Disk confocal microscope.

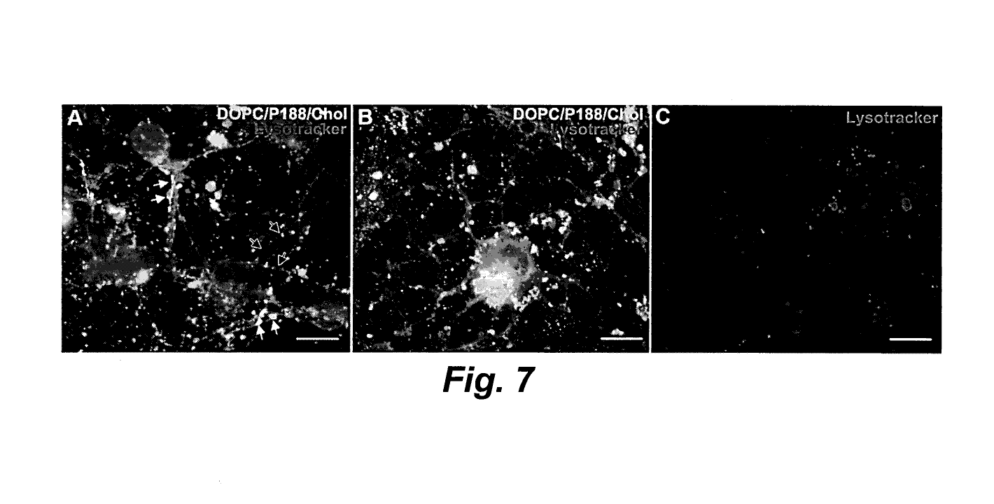

FIG. 7 shows that intense liposome-derived fluorescence was detectable along dendritic arborizations that emerge from the neuronal cell body (FIG. 7, Panel A; closed arrows), and along thin axonal processes (FIG. 7, Panel A; open arrows). Liposome-derived fluorescence is also detected in oligodendrocytes (also known as oligodendroglia), which display a complex lace-like network of processes (FIG. 7, Panel B). In both neurons (Panel A) and oliogodendrocytes (FIG. 7, Panel B), intracellularly detected fluorescent particles do not overlap with the Lysotracker fluorescence, indicating that the liposomes were not localized in the degradative lysosomes. Cells loaded with Lysotracker only are shown in FIG. 7, Panel C.

### Example 17

### Liposome Uptake by Human Neural Stem Cells

The ectoderm, one of the three primary germ cell layers in the very early embryo, differentiates to form the nervous system (brain, spine, and peripheral nerves). Further differentiation results in neural stem cells, which gives rise to neurons and glial cells. The data indicate that the neurophilic liposomes of the disclosure are taken up by neurons and glial cells, and the neurophilic liposomes uptake up by neural stem cells was investigated. Human neural stem cells were used, derived from induced pluripotent stem cell lines, to determine liposome uptake.

Dermal fibroblasts, derived from a healthy 51-year old Caucasian male with no medical problems, were used for reprogramming by the four traditional Yamanaka factors (i.e. Oct4, Sox2, Klf4, and c-Myc) to produce normal control induced pluripotent stem (iPS) cell lines. The iPS cells were then used to generate human neural stem cells according to the methods of (Xia et al., (2012) J. Mol. Neurosci. December 9. [Epub], incorporated herein by reference in its entirety).

Neural stem cells (4×10⁴) were seeded on ibidiTreat μSlides (ibidi GmbH) coated with polyornithine-laminin and cultured in NeuroCult NS-A proliferation medium (Stemcell Technologies). Three days later, neural stem cells were incubated with cyanine 5 (Cy5)-labeled DOPC/P188/Cholesterol liposomes in NeuroCult NS-A proliferation medium for 24 h at 37° C. Slides were rinsed twice with phosphate buffered saline (PBS), fixed with 4% paraformaldehyde, and mounted with Vectashield mounting medium with DAPI (Vector Laboratories). Pictures were taken using an Olympus IX81-DSU Spinning Disk confocal microscope.

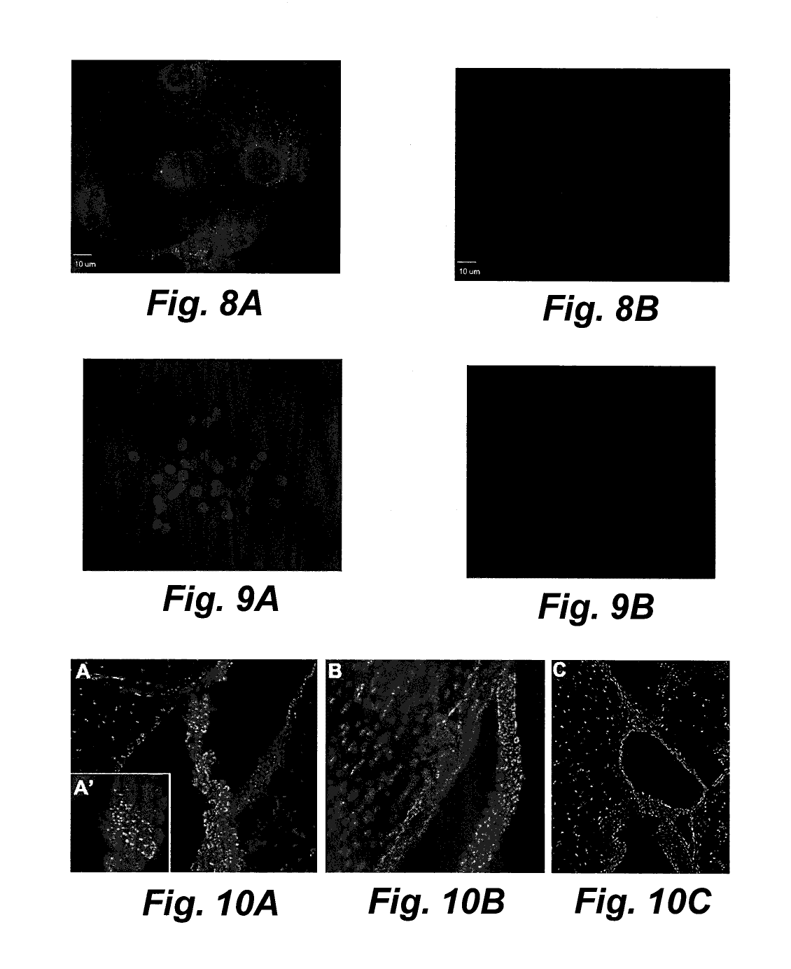

FIGS. 8A and 8B show that DOPC/P188/Cholesterol liposomes were avidly taken up by human neural stem cells. Liposome-derived fluorescence was detected as intense fluorescent particles in the cytoplasm (FIG. 8A).

### Example 18

### Liposome Uptake by Human Spinocerebellar Ataxia 2 (SCA2) Disease-Specific Neuronal Cells

It is contemplated that the neurophilic liposomes of the disclosure may be useful for delivering therapeutic drugs or imaging agents to patients with neurological diseases. To determine whether neurophilic liposomes will be taken up by diseased neuronal cells, human neurons derived from a patient with spinocerebellar ataxia 2 (SCA2) disease were used as a model. SCA2 belongs to a group of spinocerebellar ataxias, in which cerebellar ataxia (the core phenotype) is associated with extracerebellar neurological abnormalities.

Dermal fibroblasts derived from a human 30-year old male patient with SCA2 disease were used for reprogramming by the four traditional Yamanaka factors (i.e. Oct4, Sox2, Klf4, and c-Myc) to produce SCA2 disease-specific induced pluripotent stem (iPS) cell lines according to the methods of (Xia et al. 2012). SCA2 neural stem cells, generated from the iPS cells (Xia et al., (2012) J. Mol. Neurosci. December 9. [Epub]), were used to study liposome uptake. SCA2 neural stem cells, seeded in ibidiTreat μSlides coated with polyornithine-laminin, were cultured in NeuroCult NS-A proliferation medium (Stemcell Technologies). Three days later, medium was changed to Neural Induction medium (Stemcell Technologies) so that the SCA2 neural stem cells would differentiate into SCA2 neuronal cells. The next day, SCA2 neuronal cells were incubated with cyanine 5 (Cy5)-labeled DOPC/P188/Cholesterol liposomes for 24 h at 37° C. Slides were rinsed twice with phosphate buffered saline (PBS), fixed with 4% paraformaldehyde, and mounted with Vectashield mounting medium with DAPI (Vector Laboratories). Pictures were taken using an Olympus IX70 fluorescent microscope.

FIGS. 9A and 9B show that DOPC/P188/Cholesterol liposomes were taken up by neurons derived from patient with SCA2 disease (FIG. 9A). Liposome-derived fluorescence was detected as intense red fluorescent particles in the cytoplasm and axons.

### Example 19

Liposome Accumulation in Brain Choroid Plexus and Endothelia

The question of whether intravenously administered liposomes will accumulate in the brain was addressed. Cyanine 5 (Cy5)-labeled liposomes were injected into mouse tail veins. After 4 or 24 h, mice were euthanized and tissues were collected for analyses. Pictures were taken with an Olympus IX81-DSU Spinning Disk confocal microscope.

FIGS. 10A-10C show that intravenous injection of Cy5-labeled DOPC/P188/Cholesterol liposomes led to their accumulation in mouse brain choroid plexus epithelial cells and brain endothelial cells. Four h post-injection, liposome fluorescence (red) was detected in brain choroid plexus (FIG. 10A). Higher magnification of the liposome fluorescence in the choroid plexus is shown in (FIG. 10A, inset). More intense liposome fluorescence was found within the brain choroid plexus 24 h post-injection (FIG. 10B). Furthermore, intense liposome fluorescence was detected in brain endothelia (FIG. 10B).

### Example 20

### Schwann Cells and Dorsal Root Ganglion Cultures

Primary Schwann cells were prepared from neonatal rat pups and maintained as described by Notterpek et al., ((1999) Glia 25: 358-369) incorporated herein by reference in its entirety. Myelinating dorsal root ganglion (DRG) explants were established from embryonic day 12-15 mouse pups as described by Rangaraju et al., (2010) J. Neurosci. 30: 11388-11397, incorporated herein by reference in its entirety.

### Example 23

### Preparation of Celastrol-Filled Liposome Preparations

Celastrol-filled liposomes were prepared by incubating celastrol with P188 for 30 min at room temperature. Cholesterol was then added to the celastrol/P188 mixture and incubated for 10 min at room temperature. This was followed by adding DOPC to the celastrol/P188/Cholesterol mixture and incubated for another 10 min at room temperature. Tertiary butanol (3 mL) was added to the lipid drug mixture, frozen at −80° C. overnight, and lyophilized overnight. To optimize celastrol-filled liposome formulations, lipids were added to celastrol at ratios ranging between 5- to 52-fold excess by weight. Celastrol filled liposomes were stored at −20° C. until use. The day of the experiment, the lyophilizate was reconstituted with 0.9% saline. After reconstitution, unincorporated celastrol was separated from celastrol-filled liposomes by loading the lipid drug mixture onto Biospin P-30 columns (Biorad), centrifuged for 3 min, room temperature, 2000 rpm (Eppendorf Centrifuge 5804). This was followed by washing the columns with 0.5 mL PBS (5 min, 2000 rpm, Eppendorf Centrifuge 5804) twice. The column fractions were collected and concentrated by centrifuging over 10-kDa filter spin columns (Millipore) for 5 min, room temperature, 13,200 rpm (Eppendorf Centrifuge 5415D). Celastrol-filled liposomes were lysed by incubating them with DMSO (1:1, volume:volume) so that the amount of celastrol incorporated into liposomes could be quantified by measuring its absorbance at 405 nm by Biorad iMark microplate reader. Percent of celastrol incorporation was determined by measuring (amount of celastrol in liposomes after separation/amount of celastrol in liposomes before separation)×100%.

### Example 24

### Assessment of Activation of Protein Chaperone or Degradation Pathways by Celastrol

Rat Schwann cells and mouse DRG cultures were treated with unincorporated celastrol or celastrol-filled liposomes. Cells were washed, harvested, and lysed in SDS sample buffer as described by Notterpek et al., ((1999) Glia 25: 358-369). After boiling and brief centrifugation, total protein concentrations were determined and samples were analyzed by Western blotting (Notterpek et al., ((1999) Glia 25: 358-369). The bands were quantified using Scion Image software and graphed as ratios of proteins induced by unincorporated celastrol or celastrol-filled liposomes. The data indicated that celastrol was incorporated into neurophilic liposomes between 65 to 80%, when lipid weight exceeds drug weight by 15- to 25-fold.

The ability of celastrol-filled liposomes in inducing the expression of chaperone proteins, such as HSP70 and heat shock protein 27 (HSP27), by mouse Schwann cells co-incubated with mouse DRG, under myelinating conditions was determined. Free celastrol, at 250 nM but not 100 nM, induced Schwann cells to increase HSP70 expression as shown in FIG. 6B. However, celastrol-filled liposomes induced Schwann cells to increase HSP70 expression at both 100 and 250 nM concentrations (FIG. 6B). Unincorporated celastrol or celastrol-filled liposomes did not affect HSP27 expression.

Increased HSP70 levels were produced by Schwann cells, since HSP70 expression was not affected by the celastrol treatments when DRGs were depleted of Schwann cells, as shown in FIG. 6C. These data indicate that the neurophilic liposomes can enhance celastrol's ability in selectively inducing the chaperone pathway.

### Example 25

### Neurophilic Liposomes Filled with MR Contrast Agent

Gadofluorine (Bayer Schering Pharma AG), a gadolinium analogue whose chemical structure includes a perfluorinated side chain and a sugar moiety, was used. By giving rise to a bright contrast on T1-weighted MRI, gadofluorine was shown to increase the diagnostic yield of MR neurography. GadoSpin F (Gf is especially useful because since it is amphiphilic and small with molecular weight of approximately 1,300 g/mole, allowing incorporation of Gf into liposomes with high efficiency.

Preparation of Gf-Filled Liposome Preparations

Gf-filled liposomes were prepared by incubating Gf with poloxamer 188 (P188) for 30 min at room temperature. Dioleoylphosphatidylcholine (DOPC) was then added to the Gf/P188 mixture and incubated for 10 min at room temperature. This was followed by adding cholesterol (chol) to the Gf/P188/DOPC mixture and incubated for another 10 min at room temperature. Tertiary butanol (3 mL) was added to the lipid drug mixture, frozen at −80° C. overnight, and lyophilized overnight.

To optimize Gf-filled formulations, Gf was added to lipids between 15 to 75 mole percent. Gf-filled liposomes were stored at −20° C. until use. The day of the experiment, the lyophilizate was reconstituted with 0.9% saline. After reconstitution, unincorporated Gf was separated from Gf-filled liposomes by centrifuging over 10-kDa filter spin columns (Millipore) for 5 min, room temperature, 13,200 rpm. Gf incorporation was determined by Inductively-Coupled Plasma Mass Spectrometry (UF Major Analytical Instrumentation Center [MAIC]) by measuring (amount of Gf in liposomes after separation/amount of Gf in liposomes before separation)×100%.

### Example 26

### MR Imaging of Gf-Filled Liposomes

The Agilent 4.7-Tesla/200 MHz MRI spectrometer (UF AMRIS) was used to characterize the longitudinal and transverse relaxation MRI time constants of the neurophilic liposomes, filled with 45 or 60 mol % Gf, in 0.9% saline.

Various Gf-filled neurophilic liposomes, between 15 to 75 mole percent, were prepared. Unincorporated Gf was removed from Gf-filled liposomes by centrifuging over 10-kDa filter spin columns. ICP-MS was used to determine the efficiency by which Gf was incorporated into liposomes. Table 1 shows that Gf was detected in liposomes. Incorporation efficiency was about 50 to 70%. Since the actual amount of Gf found within the 60 mol % liposomes was essentially the same as that within the 75 mol % liposomes, this suggested that the maximum amount of Gf that could be incorporated into the neurophilic liposomes plateaued at 60 mol %. Liposomes filled with 45 or 60 mol % Gf were used for subsequent MR analysis.

TABLE 1
Efficiency of Gf incorporation into neurophilic liposomes

| Gf input (mol %) | Gf input (μg) | Gf incorporated (μg) | % incorporation |
|---|---|---|---|
| 15 | 4.6 | 3.1 | 64.5 |
| 30 | 9.2 | 6.3 | 65.4 |
| 45 | 13.9 | 9.2 | 67.1 |
| 60 | 18.5 | 11.4 | 61.5 |
| 75 | 23.1 | 11.5 | 49.6 |

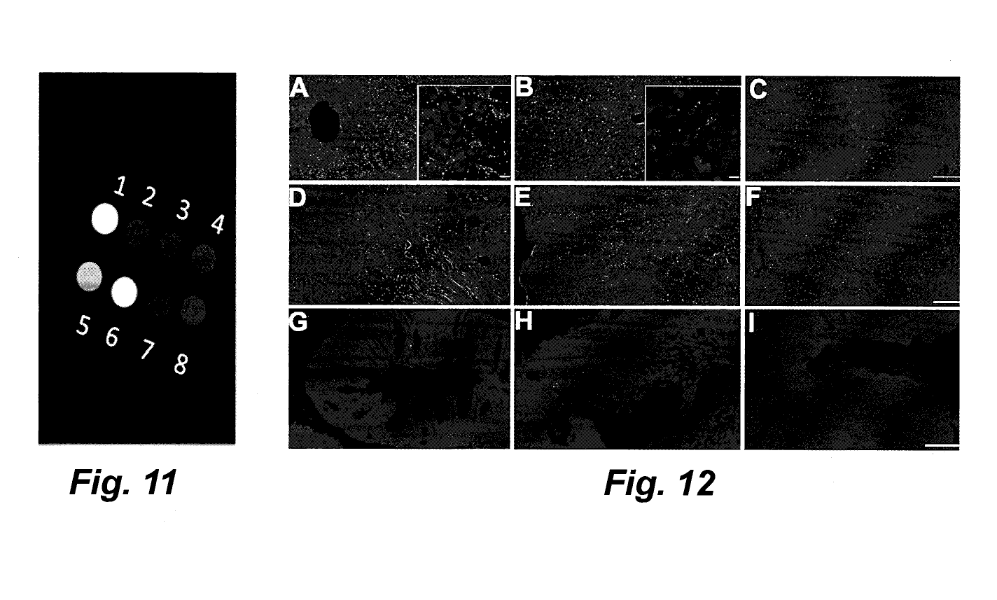

Two different Gf-filled liposomes formulations were used for the MR analysis. FIG. 11 illustrates the T1-weighted MR images of the Gf standard, various concentrations of Gf-filled liposomes, and empty liposomes (liposomes without Gf incorporation). Saline was used as negative control. As shown in FIG. 11, Gf standard produced bright T1-weighted MR image (sample 1), which is in contrast with the dim images of saline and empty liposomes (sample 8 and 7, respectively). T1-weighted MR images grew brighter with increasing concentrations of MR contrast agent. Low concentrations of liposomal Gf (sample 2: 0.0001 mM, sample 3: 0.001 mM, sample 4: 0.01 mM) also produced dim T1-weighted MR images. However higher concentrations of liposomal Gf (sample 5: 0.1 mM, sample 6; 1.0 mM) produced brighter images. In fact, the intensity of the T1-weighted MR image of 1.0 mM liposomal Gf was similar to that of the Gf standard at 1.0 mM concentration.

From the T1 and T2 measurements, we could calculate the r1 and r2 of the Gf standard and those of Gf-filled liposomes. Table 2 shows that the r1 and r2 measurements of the Gf-filled liposomes were very comparable to that of the Gf standard.

TABLE 2
r1 and r2 measurements of Gf-filled liposomes

| | r1 (mM⁻¹s⁻¹) | r2 (mM⁻¹s⁻¹) | r1/r2 |
|---|---|---|---|
| Gf Standard | 7.375 | 15.713 | 0.509 |
| Gf-filled liposomes (45 mol %) | 10.987 | 20.305 | 0.569 |
| Gf-filled liposomes (60 mol %) | 7.911 | 16.040 | 0.493 |

Gf standard and Gf-filled liposomes, at 1 mM concentration, were reconsituted in 0.9% saline. T1 and T2 of Gf standard and Gf-filled liposomes were determined by the Agilent 4.7T/200 MHz MRI spectrometer (UF ARMIS) at room temperature.

### Example 27

### Liposome Accumulation in Peripheral Nerves

The question of whether intravenously or intramuscularly administered liposomes will accumulate in the peripheral nerves was addressed. Carboxyfluorescein (CF)-labeled liposomes were injected into mouse tail vein or into mouse foot pad. After 4 h or 24 h, mice were euthanized and nerves were collected for analyses. Pictures were taken with an Olympus IX81-DSU Spinning Disk confocal microscope.

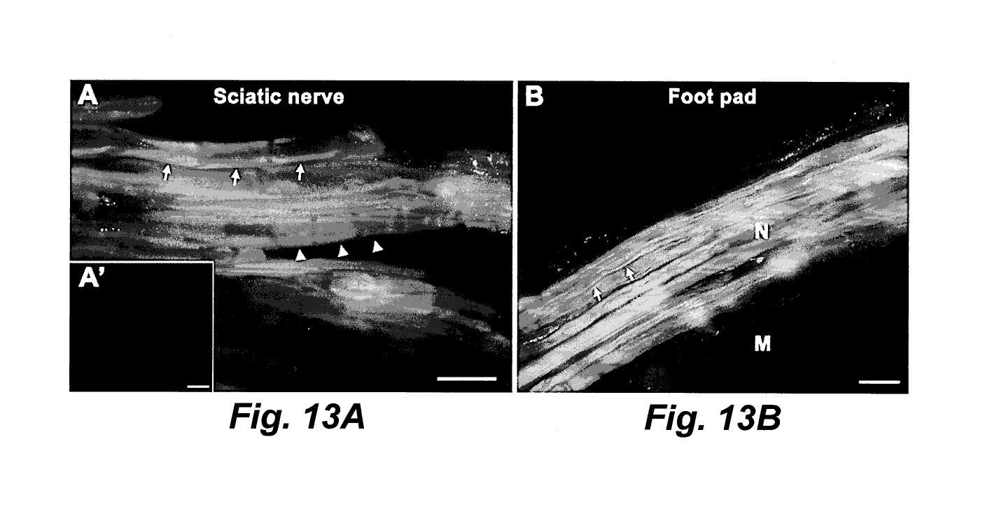

FIG. 13A shows that intravenous injection of CF-labeled DOPC/P188/Cholesterol liposomes led to their accumulation in mouse sciatic nerve. Four h post-injection, liposome fluorescence (green) was detected in the myelinated Schwann cells in the sciatic nerve (FIG. 13A). Uninjected sciatic nerve is shown in FIG. 13, inset). FIG. 13B shows that intramuscular injection of CF-labeled DOPC/P188/Cholesterol liposomes led to their preferential uptake in the nerve myelinating Schwann cells (FIG. 13B) compared to surrounding muscle tissues (FIG. 13B).

We claim:

1. A liposomal nanoparticle vehicle for the delivery of a compound to a neuronal cell or a glial cell, wherein said liposomal nanoparticle vehicle comprises:
a phospholipid;
a non-ionic surfactant; and
a cholesterol.

2. The liposomal nanoparticle vehicle of claim 1, wherein the phospholipid is selected from dioleoyl-phosphatidylcholine (DOPC), 1,2-dioleolyl-sn-glycero-3-phosphoethanolamine, or a combination thereof.

3. The liposomal nanoparticle vehicle of claim 1, wherein the non-ionic surfactant is a block copolymer.

4. The liposomal nanoparticle vehicle of claim 3, wherein the block copolymer is a tri-block copolymer having the formula (PEO)₇₅—(PPO)₃₀—(PEO)₇₅.

5. The liposomal nanoparticle vehicle of claim 1, wherein the vehicle further comprises a compound desired to be delivered to a neuronal cell or a glial cell and/or to cross a blood-brain barrier of a recipient animal or human subject.

6. The liposomal nanoparticle vehicle claim 5, wherein the compound is a detectable label, a therapeutic agent, or an imaging agent.

7. The liposomal nanoparticle vehicle of claim 6, wherein the detectable label is a fluorescent label, a detectable metal-based label, or a radiolabel.

8. The liposomal nanoparticle vehicle of claim 5, wherein the imaging agent is gadolinium, or a derivatized variant thereof.

9. The liposomal nanoparticle vehicle of claim 5, wherein the imaging agent is Gadolinium-F (Gf) or Gadolinium-M.

10. The liposomal nanoparticle vehicle of claim 5, wherein the imaging agent is Gadolinium-F (Gf).

11. The liposomal nanoparticle vehicle of claim 1, wherein the nanoparticle has a diameter between about 50 nm to about 700 nm.

12. The liposomal nanoparticle vehicle of claim 8, wherein the label is bound to a chelating agent and said chelating agent is conjugated to a lipid.

13. A method of imaging a neuronal or a glial cell or a system of neuronal/glial cells, the method comprising delivering to a recipient animal or human subject a liposomal nanoparticle vehicle according to claim 6, allowing the vehicle to deliver the detectable label or imaging agent to a neuronal or a glial cell, and detect the label in the recipient neuronal or glial cell or system of neuronal/glial cells.

14. A method of diagnosing a neuropathological condition in an animal or human subject, the method comprising delivering to the animal or human subject a liposomal nanoparticle vehicle comprising a detectable label, allowing the vehicle to access peripheral neurons or glial cells of the recipient subject, detecting the label in the peripheral nervous system of the subject, thereby determining the presence or absence of a neuromuscular pathology of the subject animal or human.

15. A method of delivering a therapeutic agent to a neuronal or a glial cell or a system of neuronal/glial cells, the method comprising contacting a recipient neuronal or glial cell or a system of neuronal/glial cells with an effective amount of a liposomal nanoparticle vehicle according to claim 6, wherein the liposomal nanoparticle vehicle comprises a therapeutic agent characterized as modulating a bioactivity of the recipient cell or tissue, allowing the vehicle to deliver the therapeutic agent to the neuronal or glial cell or system of neuronal/glial cells, whereby the therapeutic agent modulates a bioactivity of the recipient cell or tissue.

16. The method of claim 15, wherein the liposomal nanoparticle vehicle is formulated in a pharmaceutically acceptable composition.
